(19) United States
(12) Patent Application Publication
(10) Pub. No.: US 2015/0071958 A1
(43) Pub. Date: Mar. 12, 2015

(54) IMMUNOLOGICAL COMPOSITION FOR CLOSTRIDIUM DIFFICILE

(71) Applicants: Andrea Amalfitano, DeWitt, MI (US); Sergey Seregin, Okemos, MI (US)

(72) Inventors: Andrea Amalfitano, DeWitt, MI (US); Sergey Seregin, Okemos, MI (US)

(73) Assignee: Board of Trustees of Michigan State University, East Lansing, MI (US)

(21) Appl. No.: 14/361,199

(22) PCT Filed: Nov. 29, 2012
(86) PCT No.: PCT/US2012/067091

§ 371 Date: May 28, 2014

Related U.S. Application Data
(60) Provisional application No. 61/564,999, filed on Nov. 30, 2011.

(51) Int. Cl. C07K 14/33; A61K 39/08

(52) U.S. Cl. CPC C07K 14/33; A61K 39/08; A61K 38/00
USPC 424/190.1; 530/350; 530/326; 435/320.1

(57) ABSTRACT

The invention relates to methods and compositions for treating or inhibiting infection of Clostridium difficile. The methods and compositions generate rapid immune responses that inhibit future infection, and/or neutralize the toxicity caused by C. difficile infection.

This application claims benefit of the filing date of U.S. Provisional Application Ser. No. 61/564,999, filed Nov. 30, 2011, the contents of which are specifically incorporated herein by reference in their entirety.

This invention was made with government support under National Cancer Institute Grant No. U01 ES/CA 12800 awarded by the National Institutes of Health; and by the Department of Defense Grant Nos. W81XWH-07-1-0502 and W81XWH-11-1-0108 awarded by the Department of Defense. The government has certain rights in the invention.

### BACKGROUND OF THE INVENTION

Clostridium difficile (C. difficile), is a gram-positive, spore-forming, noninvasive enteric pathogen that is a leading cause of nosocomial infections in the developed world. No vaccine is currently available to prevent its infection. Life threatening manifestations of C. dificile-associated diarrhea (CDAD) include pseudomembranous colitis, toxic megacolon and systemic inflammatory response syndrome, often resulting in cardiotoxicity and heart failure. Mortality due to CDAD ranges from 6% to 30% in affected patients. More than 300,000 cases of CDAD are reported every year in the United States, and the incidence of CDAD is predicted to rise by at least 40% within the next several years. The annual cost for CDAD treatment in the U.S. is over $1.1 billion. Moreover, C. difficile is now recognized by the Center for Disease Control (CDC) as a Group II pathogen on the list of emerging and re-emerging infectious diseases by the National Institute of Allergy and Infectious Diseases (MAID). Vaccine efforts to combat C. difficile infection have been limited. Those few vaccines that have gone onto clinical trials have exhibited an inability to evoke rapid immune responses to important C. difficile antigens.

Therefore new compositions and methods for treating and inhibiting C. difficile infection are needed.

### SUMMARY OF THE INVENTION

This invention relates to methods and compositions for treating and inhibiting C. difficile infection. In some embodiments, the methods and compositions can generate rapid immune responses that inhibit future infection, and/or neutralize the toxicity caused by C. difficile infection. Such compositions and methods could be utilized both to therapeutically inhibit the establishment and progression of disease in patients recently diagnosed with C. difficile, as well as a prophylactic inhibitor for use in at-risk patients. Accordingly, the constructs described herein that expressing the C-terminal, highly immunogenic region of the C. difficile toxin A are effective vaccines against C. difficile.

One aspect of the invention is a peptide antigen with an amino acid sequence comprising at least 15 contiguous amino acids of SEQ ID NO:2, and/or with an amino acid sequence comprising 95% sequence identity to SEQ ID NO:2. For example, the peptide antigen can have an amino acid sequence comprising 95% sequence identity to any of SEQ ID NO:2, SEQ ID NO:3, SEQ ID NO:4, SEQ ID NO:7, SEQ ID NO:8, SEQ ID NO:9, SEQ ID NO:10, SEQ ID NO:11, SEQ ID NO:12, SEQ ID NO:13, SEQ ID NO:14 SEQ ID NO:15, SEQ ID NO:16, SEQ ID NO:17, SEQ ID NO:18. In some embodiments, the peptide has 96%, or 97%, or 98% or 99% sequence identity to any of SEQ ID NO:2, SEQ ID NO:3, SEQ ID NO:4, SEQ ID NO:7, SEQ ID NO:8, SEQ ID NO:9, SEQ ID NO:10, SEQ ID NO:11, SEQ ID NO:12, SEQ ID NO:13, SEQ ID NO:14 SEQ ID NO:15, SEQ ID NO:16, SEQ ID NO:17, SEQ ID NO:18. The peptide antigen can also include a combination of any of these peptide sequences.

Another aspect of the invention is an immunological composition that includes an effective amount of:
(a) a Clostridium difficile toxin A peptide with any of the sequences described herein (including a sequence selected from the group consisting of SEQ ID NO:2, SEQ ID NO:3, SEQ ID NO:4, a sequence comprising 95% sequence identity to SEQ ID NO:2, or a combination thereof);
(b) an expression vector adapted to express the Clostridium difficile toxin A peptide; or
(c) a combination thereof.

Another aspect of the invention is a method of treating or inhibiting infection of Clostridium difficile in a mammal comprising administering a composition that includes an effective amount of:
(a) a Clostridium difficile toxin A peptide with a sequence selected from the group consisting of SEQ ID NO:2, SEQ ID NO:3, SEQ ID NO:4, a sequence comprising 95% sequence identity to SEQ ID NO:2, or a combination thereof;
(b) an expression vector adapted to express the Clostridium difficile toxin A peptide; or
(c) a combination thereof.

Another aspect of the invention is an expression cassette that includes a nucleic acid encoding one of more of the peptides described herein operably linked to transcriptional regulatory element. For example, the transcriptional regulatory element can be selected from the group consisting of a promoter, an enhancer, a terminator of transcription, or a combination thereof. The expression cassette can be within a vector, such as an expression vector. The vector can be a viral vector, for example, a vector that includes a recombinant adenovirus, retrovirus, lentivirus, herpesvirus, poxvirus, papilloma virus, or adeno-associated virus. In some cases, the vector is replication incompetent viral vector such as an adenoviral vector. The vector can be incorporated into a composition.

### DESCRIPTION OF THE FIGURES

FIGS. 1A-1C show that the Adenovirus based construct that expresses a portion of the Clostridium difficile toxin A polypeptide (having SEQ ID NO:2) is able to induce rapid and robust Toxin A-specific humoral responses in mice. WT BALB/c mice were intramuscularly injected with 10¹⁰ viral particles/mouse of the Ad5-Null (n=5) or the SEQ ID NO:2 expressing Ad5-C. difficile-Toxin A (n=5) constructs. FIG. 1A shows the total Toxin A-specific IgG from plasma samples collected at 3 days post-injection. FIG. 1B shows the total Toxin A-specific IgG from plasma samples collected at 7 days post-injection. FIG. 1C shows the total Toxin A-specific IgG from plasma samples collected at 14 days post-injection. At 3, 7 and 14 days post-injection plasma samples were collected and total Toxin A-specific IgG was measured by ELISA as described in Example 1. Naïve mice (n=5) were utilized as baseline control and these values were subtracted Ad-injected values. The error bars represent ±SD. Statistical analysis was completed using two-tailed Student t-test to compare 2 groups of virally-injected animals (*—indicates p<0.05). One of two representative experiments is shown for the 14 day plasma samples.

FIGS. 2A-2B shows that the Adenovirus based construct that expresses a portion of the Clostridium difficile toxin A polypeptide (having SEQ ID NO:2) is able to induce robust Toxin A-specific T cell responses, and further illustrates that several clusters of immunogenic T cell epitopes exist in the non-enzymatic Toxin A domain. FIG. 2A shows which Toxin A peptides are immunogenic T cell epitopes as detected by the number of spot forming cells (SPFs) in an interferon-γ ELISPOT assay. Wild-type BALB/c mice were intramuscularly injected (10¹⁰ viral particles/mouse) with the Ad5-C. difficile-Toxin A (n=3) construct. Mice were sacrificed at 14 days post-injection. Splenocytes were prepared, pooled and stimulated with 2 μg/well of single peptides from a 15-mer-peptide library that spans the C. difficile Toxin A region and is encoded by the Ad5-Toxin A construct. The IFNγ ELISPOT assay was performed as described in Example 1. Naïve (n=3) pooled mice were stimulated with same individual peptides as baseline control. Six clusters of immunogenic T cell epitopes was identified (grey) in the non-enzymatic Toxin A domain. Two major immunodominant epitopes were detected as being within peptides #13 (SEQ ID NO:3) and #63 (SEQ ID NO:4). One of two representative experiments is shown. FIG. 2B shows representative pictures of wells from ELISpot assay.

FIG. 3A-3B show that the Adenovirus based construct that expresses a portion of the Clostridium difficile toxin A polypeptide (having SEQ ID NO:2) is able to induce robust Toxin A-specific T cell responses. Wild-type BALB/c mice were intramuscularly injected (10¹⁰ viral particles/mouse) with Ad5-C. difficile-Toxin A (n=5). Mice were sacrificed at 14 dpi, splenocytes were prepared, and the splenocytes were individually stimulated with pool of 12 peptides from the TA peptide library (each peptide at 0.2 μg/well). Naïve (n=5) mice were stimulated with same individual peptides as baseline control. FIG. 3A shows the results of an IFNγELISPOT assay performed as described in Example 1. FIG. 3B shows the results of an IL-2 ELISPOT assay performed as described in Example 1. One of two representative experiments is shown. Bars represent mean±SD. Statistical analysis was completed using Two-Way ANOVA with a Bonferroni post-hoc test (stimulations X treatments), p<0.05 was deemed a statistically significant difference. The symbols *, ** indicate values that are statistically different from those in naïve mice (for the same stimulation), p<0.05, p<0.001 respectively.

FIG. 4 shows that the Adenovirus based construct that expresses a portion of the Clostridium difficile toxin A polypeptide (having SEQ ID NO:2) induces robust Toxin A-specific T cell responses in comparison to the Ad5-Null vector. Wild-type BALB/c mice were intramuscularly injected (10¹⁰ viral particles/mouse) with Ad5-C. difficile-Toxin A (n=5) or Ad5-Null (n=5). Mice were sacrificed at 14 days post-injection, splenocytes were prepared, and the splenocytes were individually stimulated with 0.2 μg/well of the single most immunogenic peptides from the Toxin A library (or with inactivated Ad5 vector). IFNγ ELISPOT was performed as described in Example 1. Naïve (n=3) mice were stimulated with same peptides as baseline control. Bars represent mean±SD. Statistical analysis was completed using Two-Way ANOVA with a Bonferroni post-hoc test (stimulations X treatments), p<0.05 was deemed a statistically significant difference. The symbols *, ** indicate values, that are statistically different from those in naïve mice (for the same stimulation), p<0.05, p<0.001 respectively; #, ##—indicate significant inductions over Ad5-Null group within the same stimulation, p<0.05, p<0.001 respectively.

FIG. 5A-C show that Adenovirus based construct that expresses a portion of the Clostridium difficile toxin A polypeptide (having SEQ ID NO:2) induces pleiotropic Toxin A-specific T cell responses in contrast to Ad5-Null vector. Wild-type BALB/c mice were intramuscularly injected (10¹⁰ viral particles/mouse) with Ad5-C. difficile-Toxin A (n=5) or Ad5-Null (n=5). Mice were sacrificed at 14 days post-injection, splenocytes were prepared and the splenocytes were individually stimulated with 0.2 μg/well of the single most immunogenic peptides from the Toxin A library (or with inactivated Ad5 vector). Peptide numbers correspond to the peptide sequences shown in Table 1. Naïve (n=3) mice were stimulated with same peptides as baseline control. Bars represent mean±SD. FIG. 5A shows the results of an IL-4 ELISPOT assay performed as described in Example 1. FIG. 5B shows the results of an IL-2 ELISPOT assay performed as described in Example 1. Statistical analysis was completed using Two-Way ANOVA with a Bonferroni post-hoc test (stimulations X treatments), p<0.05 was deemed a statistically significant difference. Symbols *, ** indicate values that are statistically different from those in naïve mice (for the same stimulation), p<0.05, p<0.001 respectively; #, ##—indicate significant inductions over Ad5-Null group within the same stimulation, p<0.05, p<0.001 respectively. FIG. 5C graphically illustrates that the Ad5-Clostridium difficile-TA vaccine described herein induces robust TA-specific CD8 T cell specific responses in contrast to Ad5-Null, which does not. Wild type BALB/c mice were intramuscularly injected (10¹⁰ viral particles/mouse) with the Adenovirus based construct that expresses a portion of the Clostridium difficile toxin A polypeptide (having SEQ ID NO:2) (n=3) or with the Ad5-Null vector (n=3). Mice were sacrificed at 14 days post-infection, splenocytes were prepared, and the splenocytes were individually stimulated with 2 μg/well of peptide #63 (SEQ ID NO:4) alone or with a mixture of peptides (#9 (SEQ ID NO:10), #13 (SEQ ID NO:3), #51 (SEQ ID NO:11), #55 (SEQ ID NO:12), #63 (SEQ ID NO:4), all 0.4 μg/well). The splenocytes were stained and FACS sorted (not shown but described in Example 1). The bars represent mean±SEM. Statistical analysis was completed using a two tailed homoscedatic Student's t-test.

FIG. 6A-6B shows that Ad5-Clostridium difficile-Toxin A vaccination completely protects mice from lethal Toxin A challenge. FIG. 6A shows the percent survival of wild-type BALB/c mice that were intramuscularly injected (10¹⁰ viral particles/mouse) with Ad5-Null (▪, n=5) or with the Adenovirus based construct that expresses a portion of the Clostridium difficile toxin A polypeptide (having SEQ ID NO:2; i.e., Ad5-C. difficile-TA) (A, n=5). At 14 days post-injection mice were intraperitoneally challenged with 300 ng (6×LD₅₀) of purified toxin A, purchased from Calbiochem. Kaplan-Meyer. Survival curves are shown. Curves were compared by log-rank analysis and were found to be significantly (p<0.05) different. FIG. 6B shows the percent survival of wild-type BALB/c mice that were left uninjected (naïve, n=11) or were intramuscularly injected (10¹⁰ viral particles/mouse) with Ad5-Null (n=8) or Ad5-C. difficile-T A (n=7). At 14 days post-injection mice were intraperitoneally challenged with 300 ng (6×LD₅₀) of purified toxin A, purchased from List Biological Laboratories Inc. Kaplan-Meyer. Survival curves are shown. Curves were pair-wise compared by log-rank analysis and the following results were obtained: naïve versus Ad5-TA curve were significantly (p=0.0043) different, Ad5-Null versus Ad5-TA curves were significantly (p=0.036) different, however naïve versus Ad5-Null curves were not significantly different (p=0.486).

FIG. 7A-7B shows that Ad5-Toxin A vaccinated mice (white bars) had overall reduced portal, periportal and lobular hepatic inflammation as compared to unvaccinated mice, when challenged with toxin A. Wild-type BALB/c mice were intramuscularly injected (10¹⁰ viral particles/mouse) with Ad5-Null or Ad5-C. difficile-TA. At 14 days post-injection mice were challenged with 300 ng (6×LD₅₀) of purified toxin A (intraperitoneally injected). At 14 days post-injection survivors from both groups were sacrificed, liver sections were stained with H&E and morphometric evaluation of these sections was performed as described in Example 1. FIG. 7A shows the levels of portal, periportal and lobular inflammation in representative sections from unvaccinated censored animals (black bars), unvaccinated animals that survived (shaded bars), and Ad5-Toxin A treated animals (white bars). The levels of portal, periportal and lobular inflammation were analyzed, scored and averaged as described in Example 1. The sum of averages for each category was computed to obtain a total inflammation index score. The error bars represent ±SD. Unvaccinated censored mice (n=2) were used as positive control (high inflammation). Statistical analysis was completed using two-tailed Student t-test to compare 2 groups of virus-injected animals: Ad5-Null injected (n=4) and Ad5-TA injected (n=4). There was a trend of reduced inflammation in vaccinated mice, however, no significant differences was detected. FIG. 7B shows representative liver sections for unvaccinated censored animals, unvaccinated animals that survived, Ad5-Toxin A treated animals and naïve animals.

### DETAILED DESCRIPTION OF THE INVENTION

The invention relates to immunological compositions and methods for treating and inhibiting Clostridium difficile infection.

Clostridium difficile

Clostridium difficile (C. difficile), is a gram-positive, spore-forming, noninvasive enteric pathogen that is a leading cause of nosocomial infections in the developed world. As explained above, life threatening manifestations of C. difficile-associated diarrhea (COAD) include pseudomembranous colitis, toxic megacolon and systemic inflammatory response syndrome, often resulting in cardiotoxicity and heart failure. Mortality due to C. difficile-associated diarrhea ranges from 6% to 30% in affected patients, and more than 300,000 cases of C. difficile-associated diarrhea are reported every year in the United States. In addition, the incidence of C. difficile-associated diarrhea is predicted to rise by at least 40% within the next several years.

Mildly symptomatic or asymptomatic patients harboring C. difficile account for the majority of infectious spreading, resulting in new outbreaks. C. difficile spores can be found on environmental surfaces, equipment and clothing years after being deposited. Several host factors including advanced age, pre-existing severe illness, and broad-spectrum antibiotic usage predispose individuals to acute symptomatic C. difficile infection (Giannasca et al., Vaccine 22(7):848-56 (2004)). Recently, a new, highly virulent strain of C. difficile (BI/NAP1/r027) has been associated with outbreaks of severe nosocomial C. difficile-associated diarrhea (Ghose et al., Infect Immun. 75(6):2826-32 (2007)).

The main virulence factors of the C. difficile bacterium are the toxins A (TA) and B (TB). Both TA and TB are enteropathic and potent cytotoxic enzymes. TA and TB are also glucosyltransferases, which catalyze the inactivation of Rho proteins that are involved in cellular signaling. Together, this leads to cytotoxicity, including actin cytoskeleton depolymerization and cell death by apoptosis.

In addition, C. difficile infections induce massive cellular immune responses, including neutrophil and monocyte infiltrations, as well as cytokine and chemokine elevations, including IL-6, IL-8, IL-1β, IFNγ (Aslam et al., The Lancet infectious diseases 5(9):549-57 (2005); Hookman & Barkin, World J Gastroenterol 15(13):1554-80 (2009); Savidge et al., Gastroenterology 125(2):413-20 (2003). Moreover, following damage of the intestinal mucosa, systemic release of TA and TB from the lumen of the gut are typically observed in severe life threatening cases of C. difficile-associated diarrhea, and is correlated with acute respiratory distress syndrome, liver damage, multiple organ failure and cardiopulmonary arrest (Hamm et al., Proc Natl Acad Sci USA 103(38):14176-81 (2006); Johnson et al., Annals Intern Med 135(6):434-8 (2001); Jacob et al, Heart Lung 33(4):265-8 (2004)).

Clearly, the problem of C. difficile is a significant one, as C. difficile is now recognized by the CDC as a Group II pathogen on the NIAID list of Emerging and re-emerging infectious diseases (see website at niaid.nih.gov/topics/emerging/pages/list.aspx). A potent vaccine that can generate rapid immune responses against C. difficile infections is desirable. Such a vaccine could be utilized both as a therapeutic vaccine in patients recently diagnosed with C. difficile, as well as a prophylactic vaccine for use in at-risk patients. Vaccine efforts to combat C. difficile infection have been limited. A number of groups are working on vaccines against C. difficile infection including (Those et al., infect Immun. 75(6):2826-32 (2007); Penchine et al. Vaccine 25(20):3946-54 (2007); Sougioultzis et al. Gastroenterology 128(3):764-70 (2005); Gardiner et al., Vaccine 27(27):3598-604 (2009). However, prior to the invention, a highly efficacious vaccine has not yet been developed and such efforts do not evoke rapid immune responses to important C. difficile antigens.

Toxin A (TA) and toxin B (TB) belong to the large clostridial cytotoxin family and contain several distinct domains: (1) N-terminal enzymatic domain, (2) Central hydrophobic region, and (3) the C-terminal domain, which recognizes host cell surface carbohydrate receptors. A variety of sequences for C. difficile toxin A and toxin B are available in the database maintained by the National Center for Biotechnology Information (NCBI), which is available at the website www.ncbi.nlm.nih.gov.

One example of a C. difficile toxin A amino acid sequence is provided below as SEQ ID NO:1, and is available in the NCBI database as accession number P16154.2 (GI:1351266).

```
      1 MSLISKEELI KLAYSIRPRE NEYKTILTNL DEYNKLTTNN
    41 NENKYLQLKK LNESIDVFMN KYKTSSRNRA LSNLKKDILK
    81 EVILIKNSNT SPVEKNLHFV WIGGEVSDIA LEYIKQWADI
  121 NAEYNIKLWY DSEAFLVNTL KKAIVESSTT EALQLLEEEI
  161 QNPQFDNMKF YKKRMEFIYD RQKRFINYYK SQINKPTVPT
  201 IDDIIKSHLV SEYNRDETVL ESYRTNSLRK INSNHGIDIR
  241 ANSLFTEQEL LNIYSQELLN RGNLAAASDI VRLLALKNFG
  281 GVYLDVDMLP GIHSDLFKTI SRPSSIGLDR WEMIKLEAIM
  321 KYKKYINNYT SENFDKLDQQ LKDNFKLIIE SKSEKSEIFS
  361 KLENLNVSDL EIKIAFALGS VINQALISKQ GSYLTNLVIE
  401 QVKNRYQFLN QHLNPAIESD NNFTDTTKIF HDSLFNSATA
  441 ENSMFLTKIA PYLQVGFMPE ARSTISLSGP GAYASAYYDF
  481 INLQENTIEK TLKASDLIEF KFPENNLSQL TEQEINSLWS
  521 FDQASAKYQF EKYVRDYTGG SLSEDNGVDF NKNTALDKNY
  561 LLNNKIPSNN VEEAGSKNYV HYIIQLQGDD ISYEATCNLF
  601 SKNPKNSIII QRNMNESAKS YFLSDDGESI LELNKYRIPE
  641 RLKNKEKVKV TFIGHGKDEF NTSEFARLSV DSLSNEISSF
  681 LDTIKLDISP KNVEVNLLGC NMFSYDFNVE ETYPGKLLLS
  721 IMDKITSTLP DVNKNSITIG ANQYEVRINS EGRKELLAHS
  761 GKWINKEEAI MSDLSSKEYI FFDSIDNKLK AKSKNIPGLA
  801 SISEDIKTLL LDASVSPDTK FILNNLKLNI ESSIGDYIYY
  841 EKLEPVKNII HNSIDDLIDE FNLLENVSDE LYELKKLNNL
  881 DEKYLISFED ISKNNSTYSV RFINKSNGES VYVETEKEIF
  921 SKYSEHITKE ISTIKNSIIT DVNGNLLDNI QLDHTSQVNT
  961 LNAAFFIQSL IDYSSNKDVL NDLSTSVKVQ LYAQLFSTGL
1001 NTIYDSIQLV NLISNAVNDT INVLPTITEG IPIVSTILDG
1041 INLGAAIKEL LDEHDPLLKK ELEAKVGVLA INMSLSIAAT
1081 VASIVGIGAE VTIFLLPIAG ISAGIPSLVN NELILHDKAT
1121 SVVNYFNHLS ESKKYGPLKT EDDKILVPID DLVISEIDFN
1161 NNSIKLGTCN ILAMEGGSGH TVTGNIDHFF SSPSISSHIP
1201 SLSIYSAIGI ETENLDFSKK IMMLPNAPSR VFWWETGAVP
1241 GLRSLENDGT RLLDSIRDLY PGKFYWRFYA FFDYAITTLK
1281 PVYEDTNIKI KLDKDTRNFI MPTITTNEIR NKLSYSFDGA
1321 GGTYSLLLSS YPISTNINLS KDDLWIFNID NEVREISIEN
1361 GTIKKGKLIK DVLSKIDINK NKLIIGNQTI DFSGDIDNKD
1401 RYIFLTCELD DKISLIIEIN LVAKSYSLLL SGDKNYLISN
1441 LSNTIEKINT LGLDSKNIAY NYTDESNNKY FGAISKTSQK
1481 SIIHYKKDSK NILEFYNDST LEFNSKDFIA EDINVFMKDD
1521 INTITGKYYV DNNTDKSIDF SISLVSKNQV KVNGLYLNES
1561 VYSSYLDFVK NSDGHHNTSN FMNLFLDNIS FWKLFGFENI
1601 NFVIDKYFTL VGKTNLGYVE FICDNNKNID IYFGEWKTSS
1641 SKSTIFSGNG RNVVVEPIYN PDTGEDISTS LDFSYEPLYG
1681 IDRYINKVLI APDLYTSLIN INTNYYSNEY YPEIIVLNPN
1721 TFHKKVNINL DSSSFEYKWS TEGSDFILVR YLEESNKKIL
1761 QKIRIKGILS NTQSFNKMSI DFKDIKKLSL GYIMSNFKSF
1801 NSENELDRDH LGFKIIDNKT YYYDEDSKLV KGLININNSL
1841 FYFDPIEFNL VTGWQTINGK KYYFDINTGA ALTSYKIING
1881 KHFYFNNDGV MQLGVFKGPD GFEYFAPANT QNNNIEGQAI
1921 VYQSKFLTLN GKKYYFDNNS KAVTGWRIIN NEKYYFNPNN
1961 AIAAVGLQVI DNNKYYFNPD TAIISKGWQT VNGSRYYFDT
2001 DTAIAFNGYK TIDGKHFYFD SDCVVKIGVF STSNGFEYFA
2041 PANTYNNNIE GQAIVYQSKF LTLNGKKYYF DNNSKAVTGL
2081 QTIDSKKYYF NTNTAEAATG WQTIDGKKYY FNTNTAEAAT
2121 GWQTIDGKKY YFNTNTAIAS TGYTIINGKH FYFNTDGIMQ
2161 IGVFKGPNGF EYFAPANTDA NNIEGQAILY QNEFLTLNGK
2201 KYYFGSDSKA VTGWRIINNK KYYFNPNNAI AAIHLCTINN
2241 DKYYFSYDGI LQNGYITIER NNFYFDANNE SKMVTGVFKG
2281 PNGFEYFAPA NTHNNNIEGQ AIVYQNKFLT LNGKKYYFDN
2321 DSKAVTGWQT IDGKKYYFNL NTAEAATGWQ TIDGKKYYFN
2361 LNTAEAATGW QTIDGKKYYF NTNTFIASTG YTSINGKHFY
2401 FNTDGIMQIG VFKGPNGFEY FAPANTDANN IEGQAILYQN
2441 KFLTLNGKKY YFGSDSKAVT GLRTIDGKKY YFNTNTAVAV
2481 TGWQTINGKK YYFNTNTSIA STGYTIISGK HFYFNTDGIM
2521 QIGVFKGPDG FEYFAPANTD ANNIEGQAIR YQNRFLYLHD
2561 NIYYFGNNSK AATGWVTIDG NRYYFEPNTA MGANGYKTID
2601 NKNFYFRNGL PQIGVFKGSN GFEYFAPANT DANNIEGQAI
2641 RYQNRFLHLL GKIYYFGNNS KAVTGWQTIN GKVYYFMPDT
2681 AMAAAGGLFE IDGVIYFFGV DGVKAPGIYG
```

As described herein, the bold region of the SEQ ID NO:1 C. difficile toxin A protein (amino acids 1870-2680) is highly immunogenic. The sequence of this region is provided as SEQ ID NO:2.

1870 ALTSYKIING
1881 KHFYFNNDGV MQLGVFKGPD GFEYFAPANT QNNNIEGQAI
1921 VYQSKFLTLN GKKYYFDNNS KAVTGWRIIN NEKYYFNPNN
1961 AIAAVGLQVI DNNKYYFNPD TAIISKGWQT VNGSRYYFDT
2001 DTAIAFNGYK TIDGKHFYFD SDCVVKIGVF STSNGFEYFA
2041 PANTYNNNIE GQAIVYQSKF LTLNGKKYYF DNNSKAVTGL
2081 QTIDSKKYYF NTNTAEAATG WQTIDGKKYY FNTNTAEAAT
2121 GWQTIDGKKY YFNTNTAIAS TGYTIINGKH FYFNTDGIMQ
2161 IGVFKGPNGF EYFAPANTDA NNIEGQAILY QNEFLTLNGK
2201 KYYFGSDSKA VTGWRIINNK KYYFNPNNAI AAIHLCTINN
2241 DKYYFSYDGI LQNGYITIER NNFYFDANNE SKMVTGVFKG
2281 PNGFEYFAPA NTHNNNIEGQ AIVYQNKFLT LNGKKYYFDN
2321 DSKAVTGWQT IDGKKYYFNL NTAEAATGWQ TIDGKKYYFN
2361 LNTAEAATGW QTIDGKKYYF NTNTFIASTG YTSINGKHFY
2401 FNTDGIMQIG VFKGPNGFEY FAPANTDANN IEGQAILYQN
2441 KFLTLNGKKY YFGSDSKAVT GLRTIDGKKY YFNTNTAVAV
2481 TGWQTINGKK YYFNTNTSIA STGYTIISGK HFYFNTDGIM
2521 QIGVFKGPDG FEYFAPANTD ANNIEGQAIR YQNRFLYLHD
2561 NIYYFGNNSK AATGWVTIDG NRYYFEPNTA MGANGYKTID
2601 NKNFYFRNGL PQIGVFKGSN GFEYFAPANT DANNIEGQAI
2641 RYQNRFLHLL GKIYYFGNNS KAVTGWQTIN GKVYYFMPDT

The positions of two examples of toxin A peptides that exhibit good immunogenicity are identified in bold with underlining in the SEQ ID NO:2 sequence shown above. These two highly immunogenic peptides have the following sequences: VNGSRYYFDTDTAIA (SEQ ID NO:3) and YYFNTNTSIASTGYT (SEQ ID NO:4).

A nucleotide sequence for the SEQ ID NO:1 polypeptide is available in the NCBI database as accession number X51797.1 (GI:40439), which has SEQ ID NO:5 shown below.

```
      1 AAAGTGTTCT ATCTAATATG AAGATTTACC AATAAAAAGG
    41 TGGACTATGA TGAATGCACA GTAGTTCACC TTTTTATATT
    81 TCTAATGGTA ACAAAATATT TTTTTATATA AACCTAGGAG
  121 GCGTTATGAA TATGACAATA TCTTTTTTAT CAGAGCATAT
  161 ATTTATAAAG TTAGTAATTT TAACTATATC ATTTGATACA
  201 TTATTAGGAT GTTTAAGTGC AATAAAAAGT CGTAAATTTA
  241 ATTCTAGTTT TGGAATAGAT GGAGGAATCA GAAAAGTAGC
  281 AATGATAGCA TGTATATTTT TTTTATCAGT AGTTGACATT
  321 CTTACAAAGT TTAACTTTTT ATTTATGTTA CCACAAGATT
  361 GTATCAATTT TTTAAGACTA AAACATCTTG GAATATCTGA
  401 ATTTTTCTCT ATTTTATTTA TTTTATATGA AAGTGTAAGT
  441 ATATTAAAAA ATATGTGCTT ATGTGGATTA CCAGTACCTA
  481 AGAGATTAAA GGAAAAAATA GCAATTTTAC TAGATGCAAT
  521 GACAGATGAA ATGAATGCTA AGGATGAAAA GTAAGTAATG
  561 GTAGATATAA TAAAGATATT AACAAATAAA AAGTGTTATC
  601 CAAATAAGAA TAGCTGAAAG TTATCATAAT TCATGAAACT
  641 AATAATGAAA ACGAGGGAGC AGATGCCAAG AGACACACAA
  681 GTATTAAATA CATATAATTT CGAAGCAAGT GTTCATTACT
  721 ATATAGATGA CAAGGTAGTA TATCAAACAT TGGTTCACAA
  761 AGATGGTGCA TGGTCAGTTG GTAAAATCTA TTAAGCTACA
  801 TTAGTTACAG ATATCACAAA CTATAATAGT TAAACATAGA
  841 AATATGTGTA AATTGTGATG GAAATTATTC AAAAACACAA
  881 AAATACGTGA TGAAGGACAA AATGATATAG AAAATAAGTA
  921 TCAAACCTTA ATAAATGATT TAATTGATAG TTTAAAAGTT
  961 ATAGGAAAAA TATATAAAGA AATAAAAACA TTAAAAAAAT
1001 ATAAGATATG TTTACAAATT ACTATCAGAC AATCTCCTTA
1041 TCTAATAGAA GAGTCAATTA ACTAATTGAG TATCTTTAAA
1081 TTGAAATGTT AGGAAGTGAT TTAAATATGA AAACTTAAAT
1121 TATAAAAAAT CAATATTAAT TTATTTTTAA AAAATAGAAA
1161 GGAGTGTATA AGATTTATTT TCAAAGTTTA AAAACAAGAA
1201 AATCAATTTA AATTTCAGAA GGAATAAATG TGGTTATAGA
1241 AGTGGATTTA TTATCAAAAA TAATAATACT AGGAGGTTTT
1281 TATGTCTTTA ATATCTAAAG AAGAGTTAAT AAAACTCGCA
1321 TATAGCATTA GACCAAGAGA AAATGAGTAT AAAACTATAC
1361 TAACTAATTT AGACGAATAT AATAAGTTAA CTACAAACAA
1401 TAATGAAAAT AAATATTTGC AATTAAAAAA ACTAAATGAA
1441 TCAATTGATG TTTTTATGAA TAAATATAAA ACTTCAAGCA
1481 GAAATAGAGC ACTCTCTAAT CTAAAAAAAG ATATATTAAA
1521 AGAAGTAATT CTTATTAAAA ATTCCAATAC AAGCCCTGTA
1561 GAAAAAAATT TACATTTTGT ATGGATAGGT GGAGAAGTCA
1601 GTGATATTGC TCTTGAATAC ATAAAACAAT GGGCTGATAT
1641 TAATGCAGAA TATAATATTA AACTGTGGTA TGATAGTGAA
1681 GCATTCTTAG TAAATACACT AAAAAAGGCT ATAGTTGAAT
1721 CTTCTACCAC TGAAGCATTA CAGCTACTAG AGGAAGAGAT
1761 TCAAAATCCT CAATTTGATA ATATGAAATT TTACAAAAAA
1801 AGGATGGAAT TTATATATGA TAGACAAAAA AGGTTTATAA
1841 ATTATTATAA ATCTCAAATC AATAAACCTA CAGTACCTAC
1881 AATAGATGAT ATTATAAAGT CTCATCTAGT ATCTGAATAT
1921 AATAGAGATG AAACTGTATT AGAATCATAT AGAACAAATT
1961 CTTTGAGAAA AATAAATAGT AATCATGGGA TAGATATCAG
2001 GGCTAATAGT TTGTTTACAG AACAAGAGTT ATTAAATATT
2041 TATAGTCAGG AGTTGTTAAA TCGTGGAAAT TTAGCTGCAG
2081 CATCTGACAT AGTAAGATTA TTAGCCCTAA AAAATTTTGG
2121 CGGAGTATAT TTAGATGTTG ATATGCTTCC AGGTATTCAC
2161 TCTGATTTAT TTAAAACAAT ATCTAGACCT AGCTCTATTG
2201 GACTAGACCG TTGGGAAATG ATAAAATTAG AGGCTATTAT
2241 GAAGTATAAA AAATATATAA ATAATTATAC ATCAGAAAAC
2281 TTTGATAAAC TTGATCAACA ATTAAAAGAT AATTTTAAAC
2321 TCATTATAGA AAGTAAAAGT GAAAAATCTG AGATATTTTC
2361 TAAATTAGAA AATTTAAATG TATCTGATCT TGAAATTAAA
2401 ATAGCTTTCG CTTTAGGCAG TGTTATAAAT CAAGCCTTGA
2441 TATCAAAACA AGGTTCATAT CTTACTAACC TAGTAATAGA
2481 ACAAGTAAAA AATAGATATC AATTTTTAAA CCAACACCTT
2521 AACCCAGCCA TAGAGTCTGA TAATAACTTC ACAGATACTA
2561 CTAAAATTTT TCATGATTCA TTATTTAATT CAGCTACCGC
2601 AGAAAACTCT ATGTTTTTAA CAAAAATAGC ACCATACTTA
2641 CAAGTAGGTT TTATGCCAGA AGCTCGCTCC ACAATAAGTT
2681 TAAGTGGTCC AGGAGCTTAT GCGTCAGCTT ACTATGATTT
2721 CATAAATTTA CAAGAAAATA CTATAGAAAA AACTTTAAAA
2761 GCATCAGATT TAATAGAATT TAAATTCCCA GAAAATAATC
2801 TATCTCAATT GACAGAACAA GAAATAAATA GTCTATGGAG
2841 CTTTGATCAA GCAAGTGCAA AATATCAATT TGAGAAATAT
2881 GTAAGAGATT ATACTGGTGG ATCTCTTTCT GAAGACAATG
2921 GGGTAGACTT TAATAAAAAT ACTGCCCTCG ACAAAAACTA
2961 TTTATTAAAT AATAAAATTC CATCAAACAA TGTAGAAGAA
3001 GCTGGAAGTA AAAATTATGT TCATTATATC ATACAGTTAC
3041 AAGGAGATGA TATAAGTTAT GAAGCAACAT GCAATTTATT
3081 TTCTAAAAAT CCTAAAAATA GTATTATTAT ACAACGAAAT
3121 ATGAATGAAA GTGCAAAAAG CTACTTTTTA AGTGATGATG
3161 GAGAATCTAT TTTAGAATTA AATAAATATA GGATACCTGA
3201 AAGATTAAAA AATAAGGAAA AAGTAAAAGT AACCTTTATT
3241 GGACATGGTA AAGATGAATT CAACACAAGC GAATTTGCTA
3281 GATTAAGTGT AGATTCACTT TCCAATGAGA TAAGTTCATT
3321 TTTAGATACC ATAAAATTAG ATATATCACC TAAAAATGTA
3361 GAAGTAAACT TACTTGGATG TAATATGTTT AGTTATGATT
3401 TTAATGTTGA AGAAACTTAT CCTGGGAAGT TGCTATTAAG
3441 TATTATGGAC AAAATTACTT CCACTTTACC TGATGTAAAT
3481 AAAAATTCTA TTACTATAGG AGCAAATCAA TATGAAGTAA
3521 GAATTAATAG TGAGGGAAGA AAAGAACTTC TGGCTCACTC
3561 AGGTAAATGG ATAAATAAAG AAGAAGCTAT TATGAGCGAT
3601 TTATCTAGTA AAGAATACAT TTTTTTTGAT TCTATAGATA
3641 ATAAGCTAAA AGCAAAGTCC AAGAATATTC CAGGATTAGC
3681 ATCAATATCA GAAGATATAA AAACATTATT ACTTGATGCA
3721 AGTGTTAGTC CTGATACAAA ATTTATTTTA AATAATCTTA
3761 AGCTTAATAT TGAATCTTCT ATTGGGGATT ACATTTATTA
3801 TGAAAAATTA GAGCCTGTTA AAAATATAAT TCACAATTCT
3841 ATAGATGATT TAATAGATGA GTTCAATCTA CTTGAAAATG
3881 TATCTGATGA ATTATATGAA TTAAAAAAAT TAAATAATCT
3921 AGATGAGAAG TATTTAATAT CTTTTGAAGA TATCTCAAAA
3961 AATAATTCAA CTTACTCTGT AAGATTTATT AACAAAAGTA
4001 ATGGTGAGTC AGTTTATGTA GAAACAGAAA AAGAAATTTT
4041 TTCAAAATAT AGCGAACATA TTACAAAAGA AATAAGTACT
4081 ATAAAGAATA GTATAATTAC AGATGTTAAT GGTAATTTAT
4121 TGGATAATAT ACAGTTAGAT CATACTTCTC AAGTTAATAC
4161 ATTAAACGCA GCATTCTTTA TTCAATCATT AATAGATTAT
4201 AGTAGCAATA AAGATGTACT GAATGATTTA AGTACCTCAG
4241 TTAAGGTTCA ACTTTATGCT CAACTATTTA GTACAGGTTT
4281 AAATACTATA TATGACTCTA TCCAATTAGT AAATTTAATA
4321 TCAAATGCAG TAAATGATAC TATAAATGTA CTACCTACAA
4361 TAACAGAGGG GATACCTATT GTATCTACTA TATTAGACGG
4401 AATAAACTTA GGTGCAGCAA TTAAGGAATT ACTAGACGAA
4441 CATGACCCAT TACTAAAAAA AGAATTAGAA GCTAAGGTGG
4481 GTGTTTTAGC AATAAATATG TCATTATCTA TAGCTGCAAC
4521 TGTAGCTTCA ATTGTTGGAA TAGGTGCTGA AGTTACTATT
4561 TTCTTATTAC CTATAGCTGG TATATCTGCA GGAATACCTT
4601 CATTAGTTAA TAATGAATTA ATATTGCATG ATAAGGCAAC
4641 TTCAGTGGTA AACTATTTTA ATCATTTGTC TGAATCTAAA
4681 AAATATGGCC CTCTTAAAAC AGAAGATGAT AAAATTTTAG
4721 TTCCTATTGA TGATTTAGTA ATATCAGAAA TAGATTTTAA
4761 TAATAATTCG ATAAAACTAG GAACATGTAA TATATTAGCA
4801 ATGGAGGGGG GATCAGGACA CACAGTGACT GGTAATATAG
4841 ATCACTTTTT CTCATCTCCA TCTATAAGTT CTCATATTCC
4881 TTCATTATCA ATTTATTCTG CAATAGGTAT AGAAACAGAA
4921 AATCTAGATT TTTCAAAAAA AATAATGATG TTACCTAATG
4961 CTCCTTCAAG AGTGTTTTGG TGGGAAACTG GAGCAGTTCC
5001 AGGTTTAAGA TCATTGGAAA ATGACGGAAC TAGATTACTT
5041 GATTCAATAA GAGATTTATA CCCAGGTAAA TTTTACTGGA
5081 GATTCTATGC TTTTTTCGAT TATGCAATAA CTACATTAAA
5121 ACCAGTTTAT GAAGACACTA ATATTAAAAT TAAACTAGAT
5161 AAAGATACTA GAAACTTCAT AATGCCAACT ATAACTACTA
5201 ACGAAATTAG AAACAAATTA TCTTATTCAT TTGATGGAGC
5241 AGGAGGAACT TACTCTTTAT TATTATCTTC ATATCCAATA
5281 TCAACGAATA TAAATTTATC TAAAGATGAT TTATGGATAT
5321 TTAATATTGA TAATGAAGTA AGAGAAATAT CTATAGAAAA
5361 TGGTACTATT AAAAAAGGAA AGTTAATAAA AGATGTTTTA
5401 AGTAAAATTG ATATAAATAA AAATAAACTT ATTATAGGCA
5541 ATCAAACAAT AGATTTTTCA GGCGATATAG ATAATAAAGA
5481 TAGATATATA TTCTTGACTT GTGAGTTAGA TGATAAAATT
5521 AGTTTAATAA TAGAAATAAA TCTTGTTGCA AAATCTTATA
5561 GTTTGTTATT GTCTGGGGAT AAAAATTATT TGATATCCAA
5601 TTTATCTAAT ACTATTGAGA AAATCAATAC TTTAGGCCTA
5641 GATAGTAAAA ATATAGCGTA CAATTACACT GATGAATCTA
5681 ATAATAAATA TTTTGGAGCT ATATCTAAAA CAAGTCAAAA
5721 AAGCATAATA CATTATAAAA AAGACAGTAA AAATATATTA
5761 GAATTTTATA ATGACAGTAC ATTAGAATTT AACAGTAAAG
5801 ATTTTATTGC TGAAGATATA AATGTATTTA TGAAAGATGA
5841 TATTAATACT ATAACAGGAA AATACTATGT TGATAATAAT
5881 ACTGATAAAA GTATAGATTT CTCTATTTCT TTAGTTAGTA
5921 AAAATCAAGT AAAAGTAAAT GGATTATATT TAAATGAATC
5961 CGTATACTCA TCTTACCTTG ATTTTGTGAA AAATTCAGAT
6001 GGACACCATA ATACTTCTAA TTTTATGAAT TTATTTTTGG
6041 ACAATATAAG TTTCTGGAAA TTGTTTGGGT TTGAAAATAT
6081 AAATTTTGTA ATCGATAAAT ACTTTACCCT TGTTGGTAAA
6121 ACTAATCTTG GATATGTAGA ATTTATTTGT GACAATAATA
6161 AAAATATAGA TATATATTTT GGTGAATGGA AAACATCGTC
6201 ATCTAAAAGC ACTATATTTA GCGGAAATGG TAGAAATGTT
6241 GTAGTAGAGC CTATATATAA TCCTGATACG GGTGAAGATA
6281 TATCTACTTC ACTAGATTTT TCCTATGAAC CTCTCTATGG
6321 AATAGATAGA TATATAAATA AAGTATTGAT AGCACCTGAT
6361 TTATATACAA GTTTAATAAA TATTAATACC AATTATTATT
6401 CAAATGAGTA CTACCCTGAG ATTATAGTTC TTAACCCAAA
6441 TACATTCCAC AAAAAAGTAA ATATAAATTT AGATAGTTCT
6481 TCTTTTGAGT ATAAATGGTC TACAGAAGGA AGTGACTTTA
6521 TTTTAGTTAG ATACTTAGAA GAAAGTAATA AAAAAATATT
6561 ACAAAAAATA AGAATCAAAG GTATCTTATC TAATACTCAA
6601 TCATTTAATA AAATGAGTAT AGATTTTAAA GATATTAAAA
6641 AACTATCATT AGGATATATA ATGAGTAATT TTAAATCATT
6681 TAATTCTGAA AATGAATTAG ATAGAGATCA TTTAGGATTT
6721 AAAATAATAG ATAATAAAAC TTATTACTAT GATGAAGATA
6761 GTAAATTAGT TAAAGGATTA ATCAATATAA ATAATTCATT
6801 ATTCTATTTT GATCCTATAG AATTTAACTT AGTAACTGGA
6841 TGGCAAACTA TCAATGGTAA AAAATATTAT TTTGATATAA
6881 ATACTGGAGC AGCTTTAACT AGTTATAAAA TTATTAATGG
6921 TAAACACTTT TATTTTAATA ATGATGGTGT GATGCAGTTG
6961 GGAGTATTTA AAGGACCTGA TGGATTTGAA TATTTTGCAC
7001 CTGCCAATAC TCAAAATAAT AACATAGAAG GTCAGGCTAT
7041 AGTTTATCAA AGTAAATTCT TAACTTTGAA TGGCAAAAAA
7081 TATTATTTTG ATAATAACTC AAAAGCAGTC ACTGGATGGA
7121 GAATTATTAA CAATGAGAAA TATTACTTTA ATCCTAATAA
7161 TGCTATTGCT GCAGTCGGAT TGCAAGTAAT TGACAATAAT
7201 AAGTATTATT TCAATCCTGA CACTGCTATC ATCTCAAAAG
7241 GTTGGCAGAC TGTTAATGGT AGTAGATACT ACTTTGATAC
7281 TGATACCGCT ATTGCCTTTA ATGGTTATAA AACTATTGAT
7321 GGTAAACACT TTTATTTTGA TAGTGATTGT GTAGTGAAAA
7361 TAGGTGTGTT TAGTACCTCT AATGGATTTG AATATTTTGC
7401 ACCTGCTAAT ACTTATAATA ATAACATAGA AGGTCAGGCT
7441 ATAGTTTATC AAAGTAAATT CTTAACTTTG AATGGTAAAA
7481 AATATTACTT TGATAATAAC TCAAAAGCAG TTACCGGATT
7521 GCAAACTATT GATAGTAAAA AATATTACTT TAATACTAAC
7561 ACTGCTGAAG CAGCTACTGG ATGGCAAACT ATTGATGGTA
7601 AAAAATATTA CTTTAATACT AACACTGCTG AAGCAGCTAC
7641 TGGATGGCAA ACTATTGATG GTAAAAAATA TTACTTTAAT
7681 ACTAACACTG CTATAGCTTC AACTGGTTAT ACAATTATTA
7721 ATGGTAAACA TTTTTATTTT AATACTGATG GTATTATGCA
7761 GATAGGAGTG TTTAAAGGAC CTAATGGATT TGAATATTTT
7801 GCACCTGCTA ATACGGATGC TAACAACATA GAAGGTCAAG
7841 CTATACTTTA CCAAAATGAA TTCTTAACTT TGAATGGTAA
7881 AAAATATTAC TTTGGTAGTG ACTCAAAAGC AGTTACTGGA
7921 TGGAGAATTA TTAACAATAA GAAATATTAC TTTAATCCTA
7961 ATAATGCTAT TGCTGCAATT CATCTATGCA CTATAAATAA
8001 TGACAAGTAT TACTTTAGTT ATGATGGAAT TCTTCAAAAT
8041 GGATATATTA CTATTGAAAG AAATAATTTC TATTTTGATG
8081 CTAATAATGA ATCTAAAATG GTAACAGGAG TATTTAAAGG
8121 ACCTAATGGA TTTGAGTATT TTGCACCTGC TAATACTCAC
8161 AATAATAACA TAGAAGGTCA GGCTATAGTT TACCAGAACA
8201 AATTCTTAAC TTTGAATGGC AAAAAATATT ATTTTGATAA
8241 TGACTCAAAA GCAGTTACTG GATGGCAAAC CATTGATGGT
8281 AAAAAATATT ACTTTAATCT TAACACTGCT GAAGCAGCTA
8321 CTGGATGGCA AACTATTGAT GGTAAAAAAT ATTACTTTAA
8361 TCTTAACACT GCTGAAGCAG CTACTGGATG GCAAACTATT
8401 GATGGTAAAA AATATTACTT TAATACTAAC ACTTTCATAG
8441 CCTCAACTGG TTATACAAGT ATTAATGGTA AACATTTTTA
8481 TTTTAATACT GATGGTATTA TGCAGATAGG AGTGTTTAAA
8521 GGACCTAATG GATTTGAATA CTTTGCACCT GCTAATACGG
8561 ATGCTAACAA CATAGAAGGT CAAGCTATAC TTTACCAAAA
8601 TAAATTCTTA ACTTTGAATG GTAAAAAATA TTACTTTGGT
8641 AGTGACTCAA AAGCAGTTAC CGGACTGCGA ACTATTGATG
8681 GTAAAAAATA TTACTTTAAT ACTAACACTG CTGTTGCAGT
8721 TACTGGATGG CAAACTATTA ATGGTAAAAA ATACTACTTT
8761 AATACTAACA CTTCTATAGC TTCAACTGGT TATACAATTA
8801 TTAGTGGTAA ACATTTTTAT TTTAATACTG ATGGTATTAT
8841 GCAGATAGGA GTGTTTAAAG GACCTGATGG ATTTGAATAC
8881 TTTGCACCTG CTAATACAGA TGCTAACAAT ATAGAAGGTC
8921 AAGCTATACG TTATCAAAAT AGATTCCTAT ATTTACATGA
8961 CAATATATAT TATTTTGGTA ATAATTCAAA AGCGGCTACT
9001 GGTTGGGTAA CTATTGATGG TAATAGATAT TACTTCGAGC
9041 CTAATACAGC TATGGGTGCG AATGGTTATA AAACTATTGA
9081 TAATAAAAAT TTTTACTTTA GAAATGGTTT ACCTCAGATA
9121 GGAGTGTTTA AAGGGTCTAA TGGATTTGAA TACTTTGCAC
9161 CTGCTAATAC GGATGCTAAC AATATAGAAG GTCAAGCTAT
9201 ACGTTATCAA AATAGATTCC TACATTTACT TGGAAAAATA
9241 TATTACTTTG GTAATAATTC AAAAGCAGTT ACTGGATGGC
9281 AAACTATTAA TGGTAAAGTA TATTACTTTA TGCCTGATAC
9321 TGCTATGGCT GCAGCTGGTG GACTTTTCGA GATTGATGGT
9361 GTTATATATT TCTTTGGTGT TGATGGAGTA AAAGCCCCTG
9401 GGATATATGG CTAAAATATA TGTTTGATAA AAAATTATTC
9441 CTGTGCTACT AAGAAATTAT TTTTATATAA TAAATATTGA
9481 GATTTAATTA AAAGTCATGT GTTATTGTAA TACATGACTT
9521 TTAGTTAAAA TTTTTCTATC ATTTAATAAT CTATTATTCT
9561 TGACTATTTT ATAATAAAAT TCATATATGG AAATATTAAT
9601 ACTAAATAAT TAATAGTTGA TAAAAAATAG ATAATATGCT
9641 AAAAGCAAAA ACTAATTTAG AGCCTTGTAA CTGTTTATTT
9681 GCAATTATAA AAACATCTTT AAACATATTG ACTATAATAT
9721 AAAATATTAA CTATAATACA AAACAATATT AATTAATTTT
9761 CTCTACAGCT
```

One example of a C. difficile toxin B amino acid sequence is provided below as SEQ ID NO:6) and is available in the NCBI database as accession number CAJ67492.1 (GI:115249675).

```
      1 MSLVNRKQLE KMANVRFRTQ EDEYVAILDA LEEYHNMSEN
    41 TVVEKYLKLK DINSLTDIYI DTYKKSGRNK ALKKFKEYLV
    81 TEVLELKNNN LTPVEKNLHF VWIGGQINDT AINYINQWKD
  121 VNSDYNVNVF YDSNAFLINT LKKTVVESAI NDTLESFREN
  161 LNDPRFDYNK FFRKRMEIIY DKQKNFINYY KAQREENPEL
  201 IIDDIVKTYL SNEYSKEIDE LNTYIEESLN KITQNSGNDV
  241 RNFEEFKNGE SFNLYEQELV ERWNLAAASD ILRISALKEI
  281 GGMYLDVDML PGIQPDLFES IEKPSSVTVD FWEMTKLEAI
  321 MKYKEYIPEY TSEHFDMLDE EVQSSFESVL ASKSDKSEIF
  361 SSLGDMEASP LEVKIAFNSK GIINQGLISV KDSYCSNLIV
  401 KQIENRYKIL NNSLNPAISE DNDFNTTTNT FIDSIMAEAN
  441 ADNGRFMMEL GKYLRVGFFP DVKTTINLSG PEAYAAAYQD
  481 LLMFKEGSMN IHLIEADLRN FEISKTNISQ STEQEMASLW
  521 SFDDARAKAQ FEEYKRNYFE GSLGEDDNLD FSQNIVVDKE
  561 YLLEKISSLA RSSERGYIHY IVQLQGDKIS YEAACNLFAK
  601 TPYDSVLFQK NIEDSEIAYY YNPGDGEIQE IDKYKIPSII
  641 SDRPKIKLTF IGHGKDEFNT DIFAGFDVDS LSTEIEAAID
  681 LAKEDISPKS IEINLLGCNM FSYSINVEET YPGKLLLKVK
  721 DKISELMPSI SQDSIIVSAN QYEVRINSEG RRELLDHSGE
  761 WINKEESIIK DISSKEYISF NPKENKITVK SKNLPELSTL
  801 LQEIRNNSNS SDIELEEKVM LTECEINVIS NIDTQIVEER
  841 IEEAKNLTSD SINYIKDEFK LIESISDALC DLKQQNELED
  881 SHFISFEDIS ETDEGFSIRF INKETGESIF VETEKTIFSE
  921 YANHITEEIS KIKGTIFDTV NGKLVKKVNL DTTHEVNTLN
  961 AAFFIQSLIE YNSSKESLSN LSVAMKVQVY AQLFSTGLNT
1001 ITDAAKVVEL VSTALDETID LLPTLSEGLP IIATIIDGVS
1041 LGAAIKELSE TSDPLLRQEI EAKIGIMAVN LTTATTAIIT
1081 SSLGIASGFS ILLVPLAGIS AGIPSLVNNE LVLRDKATKV
1121 VDYFKHVSLV ETEGVFTLLD DKIMMPQDDL VISEIDFNNN
1161 SIVLGKCEIW RMEGGSGHTV TDDIDHFFSA PSITYREPHL
1201 SIYDVLEVQK EELDLSKDLM VLPNAPNRVF AWETGWTPGL
1241 RSLENDGTKL LDRIRDNYEG EFYWRYFAFI ADALITTLKP
1281 RYEDTNIRIN LDSNTRSFIV PIITTEYIRE KLSYSFYGSG
1321 GTYALSLSQY NMGINIELSE SDVWIIDVDN VVRDVTIESD
1361 KIKKGDLIEG ILSTLSIEEN KIILNSHEIN FSGEVNGSNG
1401 FVSLTFSILE GINAIIEVDL LSKSYKLLIS GELKILMLNS
1441 NHIQQKIDYI GFNSELQKNI PYSFVDSEGK ENGFINGSTK
1481 EGLFVSELPD VVLISKVYMD DSKPSFGYYS NNLKDVKVIT
1521 KDNVNILTGY YLKDDIKISL SLTLQDEKTI KLNSVHLDES
1561 GVAEILKFMN RKGNTNTSDS LMSFLESMNI KSIFVNFLQS
1601 NIKFILDANF IISGTTSIGQ FEFICDENDN IQPYFIKFNT
1641 LETNYTLYVG NRQNMIVEPN YDLDDSGDIS STVINFSQKY
1681 LYGIDSCVNK VVISPNIYTD EINITPVYET NNTYPEVIVL
1721 DANYINEKIN VNINDLSIRY VWSNDGNDFI LMSTSEENKV
1761 SQVKIRFVNV FKDKTLANKL SFNFSDKQDV PVSEIILSFT
1801 PSYYEDGLIG YDLGLVSLYN EKFYINNFGM MVSGLIYIND
1841 SLYYFKPPVN NLITGFVTVG DDKYYFNPIN GGAASIGETI
1881 IDDKNYYFNQ SGVLQTGVFS TEDGFKYFAP ANTLDENLEG
1921 EAIDFTGKLI IDENIYYFDD NYRGAVEWKE LDGEMHYFSP
1961 ETGKAFKGLN QIGDYKYYFN SDGVMQKGFV SINDNKHYFD
2001 DSGVMKVGYT EIDGKHFYFA ENGEMQIGVF NTEDGFKYFA
2041 HHNEDLGNEE GEEISYSGIL NFNNKIYYFD DSFTAVVGWK
2081 DLEDGSKYYF DEDTAEAYIG LSLINDGQYY FNDDGIMQVG
2121 FVTINDKVFY FSDSGIIESG VQNIDDNYFY IDDNGIVQIG
2161 VFDTSDGYKY FAPANTVNDN IYGQAVEYSG LVRVGEDVYY
2201 FGETYTIETG WIYDMENESD KYYFNPETKK ACKGINLIDD
2241 IKYYFDEKGI MRTGLISFEN NNYYFNENGE MQFGYINIED
2281 KMFYFGEDGV MQIGVFNTPD GFKYFAHQNT LDENFEGESI
2321 NYTGWLDLDE KRYYFTDEYI AATGSVIIDG EEYYFDPDTA
2361 QLVISE
```

In some embodiments, the toxin B peptide can have the sequence in bold above (amino acids 1750-2360). The toxin B sequence has SEQ ID NO:7, and is Shown above.

1750 I LMSTSEENKV
1761 SQVKIRFVNV FKDKTLANKL SFNFSDKQDV PVSEIILSFT
1801 PSYYEDGLIG YDLGLVSLYN EKFYINNFGM MVSGLIYIND
1841 SLYYFKPPVN NLITGFVTVG DDKYYFNPIN GGAASIGETI
1881 IDDKNYYFNQ SGVLQTGVFS TEDGFKYFAP ANTLDENLEG
1921 EAIDFTGKLI IDENIYYFDD NYRGAVEWKE LDGEMHYFSP
1961 ETGKAFKGLN QIGDYKYYFN SDGVMQKGFV SINDNKHYFD
2001 DSGVMKVGYT EIDGKHFYFA ENGEMQIGVF NTEDGFKYFA
2041 HHNEDLGNEE GEEISYSGIL NFNNKIYYFD DSFTAVVGWK
2081 DLEDGSKYYF DEDTAEAYIG LSLINDGQYY FNDDGIMQVG
2121 FVTINDKVFY FSDSGIIESG VQNIDDNYFY IDDNGIVQIG
2161 VFDTSDGYKY FAPANTVNDN IYGQAVEYSG LVRVGEDVYY
2201 FGETYTIETG WIYDMENESD KYYFNPETKK ACKGINLIDD
2241 IKYYFDEKGI MRTGLISFEN NNYYFNENGE MQFGYINIED
2281 KMFYFGEDGV MQIGVFNTPD GFKYFAHQNT LDENFEGESI
2321 NYTGWLDLDE KRYYFTDEYI AATGSVIIDG EEYYFDPDTA

The expression cassettes, expression vectors, compositions and methods provided herein can include a combination of C. difficile toxin A and toxin B peptides.
Vectors for C. difficile Peptide Expression and Delivery

Delivery vectors include, for example, viral vectors, liposomes and other lipid-containing complexes, and other macromolecular complexes capable of mediating delivery of a gene to a host cell. Vectors can also comprise other components or functionalities that further modulate gene delivery and/or gene expression, or that otherwise provide beneficial properties. Such other components include, for example, components that influence binding or targeting to cells (including components that mediate cell-type or tissue-specific binding); components that influence uptake of the vector by the cell; components that influence localization of the transferred gene within the cell after uptake (such as agents mediating nuclear localization); and components that influence expression of the gene. Such components can be provided as a natural feature of the vector (such as the use of certain viral vectors which have components or functionalities mediating binding and uptake), or vectors can be modified to provide such functionalities.

Such components also can include markers, such as detectable and/or selectable markers that can be used to detect or select for cells that have taken up and are expressing the nucleic acid delivered by the vector. Selectable markers can be encoded within a vector that expresses one or more C. difficile peptides, or such selectable markers can be encoded and/or expressed from a separate vector. Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., WO 92/08796; and WO 94/28143). Such marker genes can provide an added measure of control that can be advantageous in gene therapy contexts.

A large variety of vectors is available and can be used to express the peptides described herein. Vectors within the scope of the invention include, but are not limited to, isolated nucleic acid, vectors, e.g., recombinant adenovirus, retrovirus, lentivirus, herpesvirus, poxvirus, papilloma virus, or adeno-associated virus, including viral and non-viral vectors which are present in liposomes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes. Exemplary gene viral vectors are described below. Vectors may be administered via any route including, but not limited to, intramuscular, oral, buccal, rectal, intravenous or intracoronary administration, and transfer to cells may be enhanced using electroporation and/or iontophoresis.

In some embodiments, the vector is an adenoviral vector. Adenoviruses are medium-sized (90-100 nm), nonenveloped (naked) icosahedral viruses composed of a nucleocapsid and linear, non-segmented double stranded (ds) DNA genome which is about 36 kb long. The genes of Adenovirus are often described in terms of their expression during two phases of the adenoviral life cycle, i.e., as early phase genes or late phase genes. Genes of early phase are E1, E2, E3 and E4. Genes of late phases are L1, L2, L3, L4 and L5. The E1 gene products, including E1A and E1B, are involved in the replication of the virus. The E2 proteins provide the machinery for viral DNA replication and transcription of late genes. Most of the E3 proteins are involved in modulating the immune response of infected cells. The E4 gene products are involved in the metabolism of virus messenger RNA and provide functions that promote virus DNA replication and shut-off of host protein synthesis. One or more of these early phase genes can be deleted from an adenoviral vector employed for expression of one or more of the C. difficile peptides described herein. The virion uses its unique “spike” or fiber associated with each penton base of the capsid to attach the host cell via the coxsackie-adenovirus receptor on the surface of the host cell. There are more than fifty-three described serotypes of adenoviruses in humans.

Adenovirus type 5 has been extensively studied and can be adapted to be a vector for expression of one or more of the C. difficile peptides described herein. Recombinant adenoviral type 5 vectors usually have the E1 and E3 genes deleted from their genomes compared to wild type adenoviruses. Such deletion generates a vector that is replication defective so it can be safely used as transgene vector. For example, the E1 and E3 genes responsible for viral gene expression can be deleted from the genome to generate an adenoviral vector that is replication-incompetent. The E1 gene products, including E1A and E1B, are involved in the replication of the virus. The E3 region is not essential for in vitro viral growth. These vectors have the ability to transfect both replicating and nonreplicating cells. Adenoviral vectors can mediate transient expression of therapeutic genes in vivo, for example, peaking at seven days and lasting approximately 4 weeks. In addition, adenoviral vectors can be produced at very high titers.

The E1 plus E3 genes are about 8.0 kb and when deleted allow insertion of a nucleic acid segment that is about 8.0 kb. Hence, the coding region of the selected peptide(s), the promoter, poly A and other inserted sequences can be about 8 kb or less. For example, the promoter and poly A segments of the insert can be about 1 kb, so the maximum insert size of the segment encoding the peptide(s) may be about 7 kb. Due to higher transduction efficiency (almost 100%), higher level expression of transgene and broad range of tropism, adenovirus vector can be widely used for gene therapy, vaccine production, gene knockdown, production of membrane and hard-to-express proteins, and engineering of antibodies.

Several advantages are realized when using an Adenovirus type 5 vector with the E1 and E3 genes deleted. For example, an adenoviral vector system is a homologous system for human genes meaning that adenoviruses are human viruses, and as vectors human cells can be used as host cells. Therefore, human proteins have identical post-translational modifications as native proteins when produced be an adenoviral vector system. Adenoviral vectors have the ability to infect most mammalian cell types (both replicative and non-replicative). Adenoviral vectors accommodate reasonably large transgenes (up to 8 kb) and exhibit high expression of the recombinant protein. Adenoviral vectors can be grown at high titer (10¹⁰ viral particles/mL, which can be concentrated up to 10¹³ VP/mL). Adenoviral vectors are well tolerated, with post-infection viability of the host cells being almost 100%. Adenoviral vectors also remain epichromosomal, meaning that they do not integrate into the host chromosome and therefore do not inactivate genes or activate oncogenes. A number of vendors provide adenoviral vectors and services relating to adenoviruses such as Vector BioLabs (Philadelphia, Pa.), SignaGen Laboratories (Rockville, Md.), Microbix Biosystems Inc. (Mississauga, Ontario), and Cell Biolabs, Inc. (San Diego, Calif.).

In some embodiments, the immunological composition therefore includes C. difficile peptides encoded by and expressed from an Adenovirus (Ad) based vector. For example, the serotype 5 human adenovirus with mutations in the viral E1 and E3 genes can be used. Such an adenovirus is replication incompetent. It is a safe and highly immunogenic vector in studies with laboratory animals and early-phase human clinical trials. Wang L, et al. J Virol 83:7166-7175 (2009); Catanzaro A T, et al. Vaccine 25:4085-4092 (2007), which are herein incorporated by reference in their entireties.

One aspect of the invention is an Adenovirus based construct expressing the C-terminal, highly immunogenic region of the C. difficile toxin A (amino acids 1870-2680, e.g. SEQ ID NO:2). The results described herein indicate even moderate doses of this construct are able to generate rapid and robust C. difficile specific humoral as well as T cellular immune responses in mice. For example, the Adenovirus based construct that expresses a portion of the Clostridium difficile toxin A polypeptide (having SEQ ID NO:2) provides 100% protection from lethal challenges with toxin A.

The Adenovirus based construct can also express a Clostridium difficile toxin B peptide (e.g., a peptide selected from a region of the SEQ ID NO:6 polypeptide). Alternatively, two Adenovirus based constructs can be employed, one to express the Clostridium difficile toxin A polypeptide, and the other to express the Clostridium difficile toxin B polypeptide.

In some embodiments, the C. difficile peptide(s) are subcloned into pShuttle-CMV using procedures described by Seregin et al. (Blood 2010 Sep. 9; 116(10):1669-77 (2010), which is incorporated herein by reference in its entirety). Recombination and viral propagation can be performed as described by Seregin et al., Blood 116(10):1669-77 (2010); Ng & Graham, Methods Molec. Med. 69:389-414 (2002); Seregin et al. Gene Ther. 16(10):1245-59 (2009), each of which is specifically incorporated herein by reference in its entirety. The article by Seregin et al., Vaccine 30:1492-1501 (2012) provides further information and is specifically incorporated herein by reference in its entirety.

### Dosages and Dosage Forms

The amounts of vector(s) or peptides administered to achieve a particular outcome will vary depending on various factors including, but not limited to, the gene and promoter chosen, the condition, patient specific parameters, e.g., height, weight and age, and whether prevention or treatment is to be achieved. The vector or peptide may be amenable to chronic use.

The vectors and/or peptides described herein can be used to protect against or ameliorate the symptoms of Clostridium difficile associated diarrhea (CDAD). As illustrated herein, an immunological composition that includes the peptides and/or vectors described herein has efficacy for treating and inhibiting infection of C. difficile. For example, the vectors and/or peptides described herein can induce robust Toxin A-specific T cell responses. The Toxin A peptides described herein, whether injected as immunogens or administered and expressed from an expression vector, are immunogenic T cell epitopes. In addition, vaccination with the vectors and/or peptides described herein completely protects mammals from lethal exposure to Toxin A.

Vectors or peptides of the invention may conveniently be provided in the form of formulations suitable for administration. A suitable administration format may best be determined by a medical practitioner for each patient individually, according to standard procedures. Suitable pharmaceutically acceptable carriers and their formulation are described in standard formulations treatises, e.g., Remington's Pharmaceuticals Sciences. Vectors or fusion peptides of the present invention may be formulated in solution at neutral pH, for example, about pH 6.5 to about pH 8.5, more preferably from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, more preferably from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is preferred particularly for buffers containing sodium ions. If desired, solutions of the above compositions can also be prepared to enhance shelf life and stability. Therapeutically useful compositions of the invention can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

The vectors or peptides can be provided in a dosage form containing an amount of a vector or peptide effective in one or multiple doses. For viral vectors, the effective dose may be in the range of at least about 10⁷ viral particles, e.g., about 10⁹ viral particles, 10¹¹ viral particles or 10¹⁴ viral. As noted, the exact dose to be administered is determined by the attending clinician, but is may be in 1 mL phosphate buffered saline. For delivery of plasmid DNA alone, or plasmid DNA in a complex with other macromolecules, the amount of DNA to be administered will be an amount which results in a beneficial effect to the recipient. For example, from 0.0001 to 1 mg or more, e.g., up to 1 g, in individual or divided doses, e.g., from 0.001 to 0.5 mg, or 0.01 to 0.1 mg, of DNA can be administered. For delivery of the peptide, the amount administered is an amount which results in a beneficial effect to the recipient. For example, from 0.0001 to 100 mg or more, e.g., up to 1 g, in individual or divided doses, e.g., from 0.001 to 0.5 g, or 0.01 to 0.1 g, of peptide can be administered.

Administration of the vector or peptide in accordance with the present invention may be continuous or intermittent, depending, for example, upon the recipient's physiological condition, whether the purpose of the administration is therapeutic or prophylactic, and other factors known to skilled practitioners. The administration of the vector or peptide may be essentially continuous over a preselected period of time or may be in a series of spaced doses. Both local (e.g., intramuscular) and systemic administration is contemplated.

One or more suitable unit dosage forms comprising the vector or peptide, which may optionally be formulated for sustained release, can be administered by a variety of routes including oral, or parenteral, including by rectal, buccal, vaginal and sublingual, transdermal, subcutaneous, intravenous, intramuscular, intraperitoneal, intrathoracic, intrapulmonary and intranasal routes. In some embodiments, administration can be into the blood stream (e.g., in an intracoronary artery). The formulations may, where appropriate, be conveniently presented in discrete unit dosage forms and may be prepared by any of the methods well known to pharmacy. Such methods may include the step of bringing into association the vector or peptide with liquid carriers, solid matrices, semi-solid carriers, finely divided solid carriers or combinations thereof, and then, if necessary, introducing or shaping the product into the desired delivery system.

Pharmaceutical formulations containing the vector or peptide can be prepared by procedures known in the art using well known and readily available ingredients. For example, the agent can be formulated with common excipients, diluents, or carriers, and formed into tablets, capsules, suspensions, powders, and the like. The vectors or peptides of the invention can also be formulated as elixirs or solutions for convenient oral administration or as solutions appropriate for parenteral administration, for instance by intramuscular, subcutaneous or intravenous routes.

The pharmaceutical formulations of the vectors or peptides can also take the form of an aqueous or anhydrous solution or dispersion, or alternatively the form of an emulsion or suspension.

Thus, the vector or peptide(s) may be formulated for parenteral administration (e.g., by injection, for example, bolus injection or continuous infusion) and may be presented in unit dose form in ampoules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint.

For administration to the upper (nasal) or lower respiratory tract by inhalation, the vectors or peptide(s) are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the therapeutic agent (e.g., a vector or peptide) and a suitable powder base such as lactose or starch. The powder composition may be presented in unit dosage form in, for example, capsules or cartridges, or, e.g., gelatin or blister packs from which the powder may be administered with the aid of an inhalator, insufflator or a metered-dose inhaler.

For intra-nasal administration, the vectors or peptides may be administered via nose drops, a liquid spray, such as via a plastic bottle atomizer or metered-dose inhaler. Typical of atomizers are the Mistometer (Wintrop) and the Medihaler (Riker).

For topical administration, the vectors or peptides may be formulated as is known in the art for direct application to a target area. Conventional forms for this purpose include wound dressings, coated bandages or other polymer coverings, ointments, creams, lotions, pastes, jellies, sprays, and aerosols, as well as in toothpaste and mouthwash, or by other suitable forms. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active ingredients can also be delivered via iontophoresis, e.g., as disclosed in U.S. Pat. No. 4,140,122; 4,383,529; or 4,051,842. The percent by weight of a therapeutic agent of the invention present in a topical formulation will depend on various factors, but generally will be from 0.01% to 95% of the total weight of the formulation, and typically 0.1-25% by weight.

When desired, the above-described formulations can be adapted to give sustained release of the active ingredient employed, e.g., by combination with certain hydrophilic polymer matrices, e.g., comprising natural gels, synthetic polymer gels or mixtures thereof.

Drops, such as eye drops or nose drops, may be formulated with an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents drop-wise, via a specially shaped closure.

The vectors or peptides may further be formulated for topical administration in the mouth or throat. For example, the active ingredients may be formulated as a lozenge further comprising a flavored base, usually sucrose and acacia or tragacanth; pastilles comprising the composition in an inert base such as gelatin and glycerin or sucrose and acacia; mouthwashes comprising the composition of the present invention in a suitable liquid carrier; and pastes and gels, e.g., toothpastes or gels, comprising the composition of the invention.

The formulations and compositions described herein may also contain other ingredients such as antiviral agents, antimicrobial agents, anti-inflammatory agents or preservatives.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating the vector or peptide in a selected amount in the appropriate solvent with various of the other ingredients enumerated above, as required, followed by filtered sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preparation can be vacuum dried or freeze dried to yield a powder of the active ingredient plus any additional desired ingredient from the previously sterile-filtered solution thereof.

For purposes of topical administration, dilute sterile, aqueous solutions (usually in about 0.1% to 5% concentration), otherwise similar to the above parenteral solutions, are prepared in containers suitable for incorporation into a transdermal patch, and can include known carriers, such as pharmaceutical grade dimethylsulfoxide (DMSO).

The therapeutic vectors and/or peptides of this invention may be administered to a mammal alone or in combination with pharmaceutically acceptable carriers. As noted above, the relative proportions of active ingredient and carrier are determined by the solubility and chemical nature of the compound, chosen route of administration and standard pharmaceutical practice.

The dosage of the present therapeutic agents which will be most suitable for prophylaxis or treatment will vary with the form of administration, and the physiological characteristics of the particular patient under treatment. Generally, small dosages will be used initially and, if necessary, will be increased by small increments until the optimum effect under the circumstances is reached.

### DEFINITIONS

A “vector” refers to a macromolecule or association of macromolecules that comprises or associates with a polynucleotide, and which can be used to mediate delivery of the polynucleotide to a cell, either in vitro or in vivo. Illustrative vectors include, for example, plasmids, viral vectors, liposomes and other gene delivery vehicles. The polynucleotide to be delivered, sometimes referred to as a “target polynucleotide” or “transgene,” may comprise a coding sequence of interest in gene therapy (such as a gene encoding a protein of therapeutic interest), a coding sequence of interest in vaccine development (such as a polynucleotide expressing a protein, polypeptide or peptide suitable for eliciting an immune response in a mammal), and/or a selectable or detectable marker.

“Transduction,” “transfection,” “transformation” or “transducing” as used herein, are terms referring to a process for the introduction of an exogenous polynucleotide, e.g., a transgene in vector, into a host cell leading to expression of the polynucleotide, e.g., the transgene in the cell, and includes the use of recombinant virus to introduce the exogenous polynucleotide to the host cell. Transduction, transfection or transformation of a polynucleotide in a cell may be determined by methods well known to the art including, but not limited to, protein expression (including steady state levels), e.g., by ELISA, flow cytometry and Western blot, measurement of DNA and RNA by heterologous hybridization assays, e.g., quantitative polymerase chain reaction (qPCR), Northern blots, Southern blots and gel shift mobility assays. Methods used for the introduction of the exogenous polynucleotide include well-known techniques such as viral infection or transfection, lipofection, transformation and electroporation, as well as other non-viral gene delivery techniques. The introduced polynucleotide may be stably or transiently maintained in the host cell.

“Gene expression” or “expression” refers to the process of gene transcription, translation, and post-translational modification.

The term “nucleic acid” or “polynucleotide” refers to a polymeric form of nucleotides of any length, including deoxyribonucleotides or ribonucleotides, or analogs thereof. A nucleic acid may comprise modified nucleotides, such as methylated or capped nucleotides and nucleotide analogs, and may be interrupted by non-nucleotide components. If present, modifications to the nucleotide structure may be imparted before or after assembly of the polymer. The term nucleic acid, as used herein, refers interchangeably to double- and single-stranded molecules. Unless otherwise specified or required, any embodiment of the invention described herein that is a nucleic acid encompasses both the double-stranded form and each of two complementary single-stranded forms known or predicted to make up the double-stranded form.

A “transcriptional regulatory element” refers to a nucleic acid segment that controls the transcription of a gene or coding sequence to which it is operably linked. Transcriptional regulatory sequences of use in the present invention generally include at least one transcriptional promoter and may also include one or more enhancers and/or terminators of transcription (e.g., a CMV/CMV regulatory cassette).

“Operably linked” refers to an arrangement of two or more components, wherein the components so described are in a relationship permitting them to function in a coordinated manner. By way of illustration, a transcriptional regulatory sequence or a promoter is operably linked to a coding sequence if the transcriptional regulatory sequence or promoter promotes transcription of the coding sequence. An operably linked transcriptional regulatory sequence is generally joined in cis with the coding sequence, but it is not necessarily directly adjacent to it.

“Heterologous” means derived from a genotypically distinct entity from the entity to which it is compared. For example, a polynucleotide introduced by genetic engineering techniques into a different cell type is a heterologous nucleic acid (and, when expressed, can encode a heterologous polypeptide). Similarly, a transcriptional regulatory element such as a promoter that is removed from its native coding sequence and operably linked to a different coding sequence is a heterologous transcriptional regulatory element.

A “terminator” refers to a nucleic acid sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical examples of such sequence-specific terminators include polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators), and may be comprised of sequence-specific termination sequences or sequence-non-specific terminators or both. A variety of such terminator sequences are known in the art; and illustrative uses of such sequences within the context of the present invention are provided below.

“Host cells,” “cell lines,” “cell cultures,” “packaging cell line” and other such terms denote higher eukaryotic cells, such as mammalian cells including human cells, useful in the present invention, e.g., to produce recombinant virus or recombinant peptide. These cells include the progeny of the original cell that was transduced. It is understood that the progeny of a single cell may not necessarily be completely identical (in morphology or in genomic complement) to the original parent cell.

“Recombinant,” as applied to a nucleic acid means that the nucleic acid is the product of various combinations of cloning, restriction and/or ligation steps, and other procedures that result in a construct that is distinct from a nucleic acid found in nature. A recombinant virus is a viral particle comprising a recombinant nucleic acid. The term includes replicates of the original polynucleotide construct and progeny of the original virus construct.

A “control element” or “control sequence” is a nucleic acid sequence involved in an interaction of molecules that contributes to the functional regulation of a nucleic acid, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter. Promoters include adenovirus promoters, AAV promoters, e.g., P5, P19, P40 and AAV ITR promoters, as well as heterologous promoters.

An “expression vector” is a vector comprising a region which encodes a peptide or gene product of interest, and is used for effecting the expression of the peptide or gene product in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are available in the art or can be readily constructed from components that are available in the art.

The terms “polypeptide” and “protein” are used interchangeably herein to refer to polymers of amino acids of any length. The terms also encompass an amino acid polymer that has been modified; for example, disulfide bond formation, glycosylation, acetylation, phosphorylation, lipidation, or conjugation with a labeling component.

An “isolated” product, e.g., plasmid, virus, nucleic acid, polypeptide or other substance refers to a preparation of the product devoid of at least some of the other components that are typically present when that product is in its natural form. Thus, for example, an isolated product may be prepared by using a purification technique to enrich it from a source mixture. Enrichment can be measured on an absolute basis, such as weight per volume of solution, or it can be measured in relation to a second, potentially interfering substance present in the source mixture. In some embodiments, increasing enrichments of a product are more preferred. Thus, for example, a 2-fold enrichment is preferred, 10-fold enrichment is more preferred, 100-fold enrichment is more preferred, 1000-fold enrichment is even more preferred.

The invention will be described by the following nonlimiting examples.

### Example 1

### Materials and Methods

This Example describes some of the materials and methods that have been used in the development of the invention.

Adenovirus Vector Construction, Production and Characterization:

All Adenoviruses utilized in this study were human Ad type 5-derived replication deficient vectors (deleted for the E1 and E3 genes). The Ad5-TA construct was made by specifically selecting a TA sequence that had not previously been tested, and optimizing the DNA encoding this TA sequence for human expression. The synthetic gene was obtained from GeneArt (Regensburg Germany, see website at geneart.com; now part of Life Technologies Corporation). The C-terminal region of TA (spanning amino acids 1870-2680) was subcloned into pShuttle-CMV using procedures described by Seregin et al. (Blood 116(10):1669-77 (2010)). This C terminal domain of TA is non-toxic and lacks enzymatic activity. Recombination and viral propagation were completed as described by Seregin et al. (2010); Ng & Graham, Methods Molec. Med. 69:389-414 (2002); Seregin et al. Gene Ther. 16(10):1245-59 (2009)). An Ad5-Null vector was constructed by recombining pShuttle (with no transgene) with pAdEasyl and purifying the vector construct as described by Ng & Graham (Methods Molec. Med. 69:389-414 (2002)). Propagation and characterization of all Ads was performed as previously described by Seregin et al. (Blood 116(10):1669-77 (2010)). All viruses were found to be free of replication-competent adenoviruses (RCA) both by polymerase chain reaction (PCR) of a RCA-specific region (E1 region amplification) and direct sequencing methods as previously described in Seregin et al., (Mol Ther 17(4):685-96 (2009)). All Ads were also tested for the presence of bacterial endotoxin as previously described by Seregin et al., (Mol Ther 17(4):685-96 (2009)), and were found to contain <0.15 EU endotoxin per ml.

Animal Procedures:

Adult BALB/c WT mice were purchased from Jackson Laboratory (Bar Harbor, Me.). Ad5 vectors were injected intramuscularly (IM), into the tibialis anterior of the right hindlimb, total volume 25 μl) into 8 weeks old male mice after performing proper anesthesia with isofluorane. A total of 1×10¹⁰ virus particles per mouse were administered IM. The number of animals used for each experiment was noted and is specified in the description of the figure(s). Toxin A challenge experiments were performed at 14 days post-injection as previously described by Gardiner et al. (Vaccine 27(27):3598-604 (2009)). In particular, 300 ng of freshly reconstituted toxin A (List Biological Laboratories Inc., Campbell, Calif. or Calbiochem, San Diego, Calif.) in 100 μl (in PBS) was injected intraperitoneally per mouse. After challenge the mice were carefully and routinely monitored every 6 hours by lab personnel and by technicians from MSU animal core facility for mortality and other parameters, all in accordance with MSU ORCBS and IACUC. Plasma and tissue samples were collected and processed at the indicated time points in accordance with Michigan State University Institutional Animal Care and Use Committee. All procedures with recombinant Ads and TA were performed under BSL-2, and all vector-treated animals were maintained in ABSL-2 conditions. All animal procedures were reviewed and approved by the Michigan State University ORCBS and IACUC. Care for mice was provided in accordance with PHS and AAALAC standards.

Antibody Titering Assay:

ELISA based titering experiments were essentially completed as previously described by Gardiner et al. (Vaccine 27(27):3598-604 (2009); Seregin et al. Gene Ther. 16(10):1245-59 (2009); and Hensley et al. Mol. Ther. 15(2):393-403 (2007)). Briefly, 50-100 ng of purified toxin A (diluted in PBS) was used to coat wells of a 96 well plate overnight at 4° C. Plates were washed with PBS-Tween (0.05%) solution, and blocking buffer (3% BSA in PBS) was added to each well and incubated for 1-3 hours at room temperature. Total IgG antibodies, were measured in plasma samples collected from naïve, Ad5-Null or Ad5-TA injected mice. Collection of plasma samples was performed at 3, 7 or 14 dpi. Dilutions were made in blocking buffer (1:10 to 1:40,000). Following dilution, plasma was added to the wells, and incubated at room temperature for 1 hour. Wells were washed using PBS-Tween (0.05%) and HRP-conjugated rabbit anti-mouse antibodies (BioRad, Hercules, Calif.) were added at a 1:5000 dilution in PBS-Tween. TMB (Sigma-Aldrich, St. Louis, Mo.) substrate was added to each well, and the reaction was stopped with 2 N sulfuric acid. Plates were read at 450 nm in a microplate spectrophotometer.

ELISpot Analysis:

96-well Multiscreen high protein binding Immobilon-P membrane plates (Millipore, Billerica, Mass.) were pre-treated with ethanol, coated with mouse anti-IFNγ (or IL-4, or IL-2) capture antibody, incubated overnight, and blocked with RPMI medium (with 10% FBS, 1% PSF) prior to the addition of 1.0×10⁶ splenocytes/well (see, Seregin et al., Blood 116(10):1669-77 (2010); Weaver & Barry, Hum Gene Ther (September 2008)). Ex vivo stimulation included the incubation of splenocytes for 18-24 hours in a 37° C., 5% CO₂ incubator in 100 μL of media alone (unstimulated), or in 100 μL media containing:

(1) 2 μg/well of single peptides from a 15-mer-peptide library, spanning the C. difficile TA region, encoded by Ad-TA vaccine (15 amino acid peptides overlapping by 5 amino acids on both N- and C-termini); or

(2) a pool of 12 peptides from the library (each peptide 0.2 μg/well); or

(3) 0.2 μg/well of single most immunogenic peptides from the library; or

(4) Ad5-null vector inactivated at 56° C. for 45 min (100 viral particles/cell).

The library was produced by JPT Peptide Technologies (Berlin, Germany; see website at jpt.com). Ready-set Go IFNγ, IL-2 and IL-4 mouse ELISpot kits were purchased from eBioscience (San Diego, Calif.) Staining of plates was completed per the manufacturer's protocol. Spots were counted and photographed by an automated ELISpot reader system (Cellular Technology, Cleveland, Ohio).

Cell Staining and Flow Cytometry:

Splenocytes from immunized mice were ex vivo stimulated with peptide #63 (2 μg/well) or with mixture of peptides (#9, #13, #51, #55, #63, all 0.4 μg/well) where the sequences of peptides are shown in Table. Following stimulation splenocytes were stained with the following antibodies: PerCpCy5.5-CD3, Alexa Floure700-CD8a, PE-Cy7-CD4, FITC-IFNγ, PE-IL2, Alexa Fluor647-IL4 (4 μg/ml), all obtained from BD Biosciences (San Diego, Calif.). Cells were incubated on ice with the respective antibodies for surface staining for 30 min. After washing, intracellular staining was performed: cells were fixed with 2% formaldehyde (Polysciences, Warrington, Pa.), permeabilized with 0.2% Saponin (Sigma-Aldrich, St. Louis, Mo.), and stained. The violet fluorescent reactive dye (ViViD, Invitrogen) was included as a viability marker to exclude dead cells from the analysis. Cells were sorted using an LSR II instrument and analyzed using FlowJo software (Aldhammen et al., J Immunol 186((2):722-32 (January 2011)).

Hematoxylin and Eosin Staining (Hepatic Inflammation):

Upon sacrifice, liver tissues were fixed in 10% neutral buffer formalin for 12 hours, washed in 70% ethanol, embedded in paraffin and 6-μm sectioned were stained with H&E, exactly as previously described (Seregin et al., Mol Ther 17(4):685-96 (2009); Hu et al., Hum Gene Ther 10(3):355-64 (1999)). The inventors adapted a previously developed semi-quantitative scoring system, which allows the level of hepatic pathology between different liver sections to be quantified and statistically compared (Seregin et al., Mol Ther 17(4):685-96 (2009); Hu et al., Hum Gene Ther 10(3):355-64 (1999)). For every mouse, liver sections obtained at different portions of the liver (0-1000 μm from liver surface) were analyzed and given a numerical score (0-3) for three different categories of liver pathology (portal, periportal, lobular) for at least 15 fields per mouse. A qualified pathologist was consulted prior to performing scoring in blind manner (performed by 2 researchers with similar results). The sum of scores (all fields) for each mouse was taken and individual category scores were averaged for each group. Total inflammation index was computed by averaging the sum of all three individual category scores for each mouse (Seregin et al., Mol Ther 17(4):685-96 (2009); Hu et al., Hum Gene Ther 10(3):355-64 (1999)).

Statistical Analysis:

For every experiment, pilot trials were performed with N=3 per group. This allowed us to determine effect size and sample variance so that Power Analysis could be performed to correctly determine the number of subjects per group required to achieve a statistical Power >0.8 at the 95% confidence level. Statistically significant differences in Clostridium difficile specific adaptive immune responses were determined using statistical analyses specified in figure legends. Kaplan Meier survival analysis was performed for challenge experiments. Graphs in this paper are presented as Mean of the average±SD, unless otherwise specified. GraphPad Prism software was utilized for statistical analysis.

### Example 2

### Adenovirus Based Vaccine Against Clostridium difficile Toxin A is Able to Induce Rapid and Robust Toxin A-Specific Humoral Responses in Mice

This Example illustrates the immunogenicity of the toxin A peptides with a SEQ ID NO: 2 sequence, including peptide sequences VNGSRYYFDTDTAIA (SEQ ID NO:3) and YYFNTNTSIASTGYT (SEQ ID NO:4).

### IgG Antibody Production Versus the SEQ ID NO:2 Toxin A Peptide

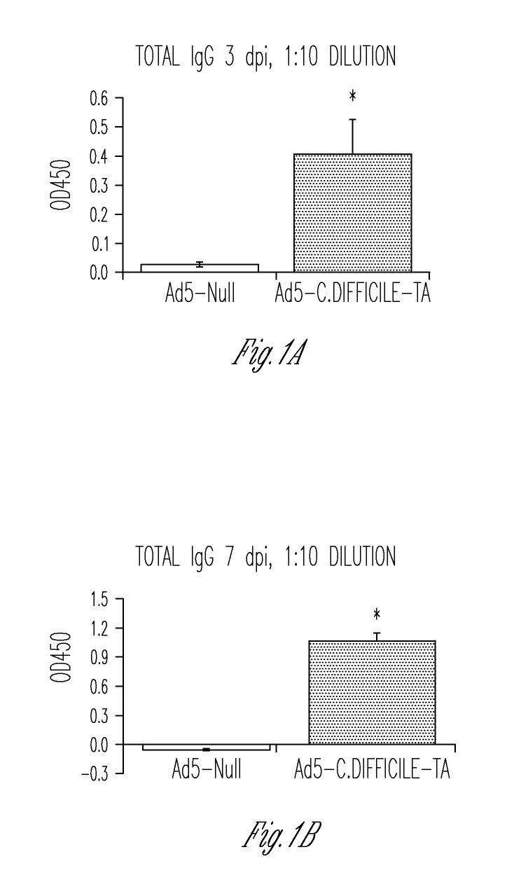

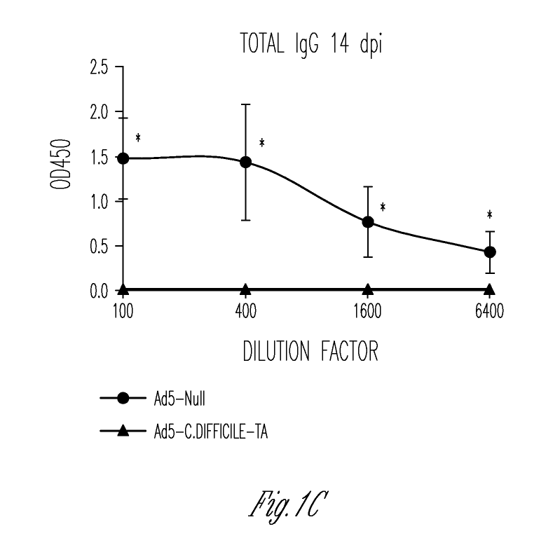

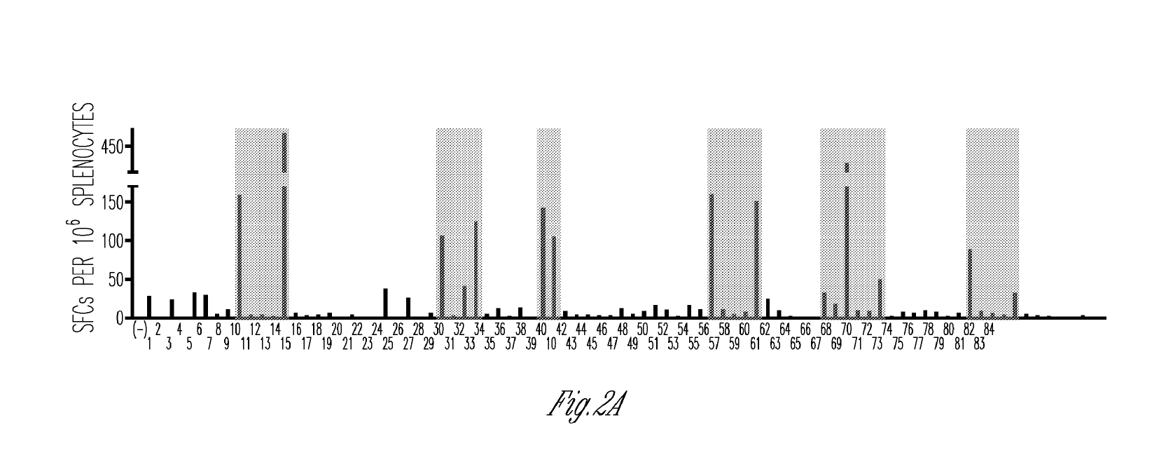

As described in Example 1, a recombinant, E1 and E3 gene deleted Ad based construct was modified to express the C-terminal, highly immunogenic region of C. difficile toxin A (amino acids 1870-2680; e.g., SEQ ID NO:2). This vaccine construct is referred to herein as Ad5-TA (or Ad5-C. difficile-T A). Humoral responses are important for protection from C. difficile-associated diarrhea (CDAD1 To investigate if an Ad5-TA vaccine construct is able to induce C. difficile specific humoral responses, the Ad5-TA construct was intramuscularly (IM) injected into adult BALB/c mice. A with moderate dose (10¹⁰ viral particles/mouse) of Ad5-TA or a control construct was employed. The control construct is referred to as Ad5-Null and is also described in Example 1. Elevated plasma levels of TA-specific antibodies were detected at 3, 7 and 14 days post injection (dpi) of the Ad5-TA construct. More specifically, significant (p<0.05) IgG titers were present as early as 3 dpi (FIG. 1A), IgG titers were further increased by 7 dpi (FIG. 1B) and IgG titers were further increased by 14 dpi (FIG. 1C; some supporting data not shown).

### Ad5-TA and Peptides Therein Elicit Robust Toxin A-Specific T Cell Responses

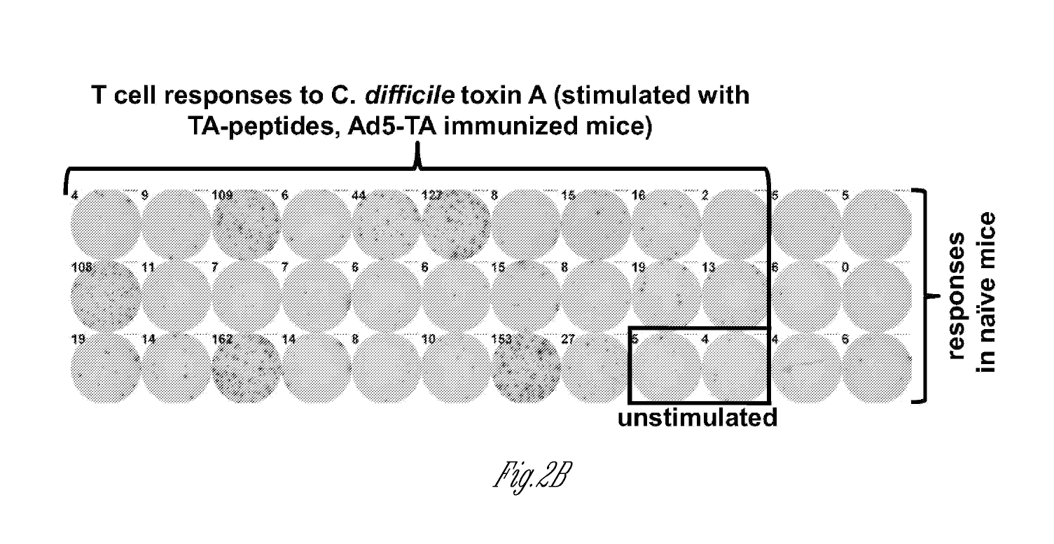

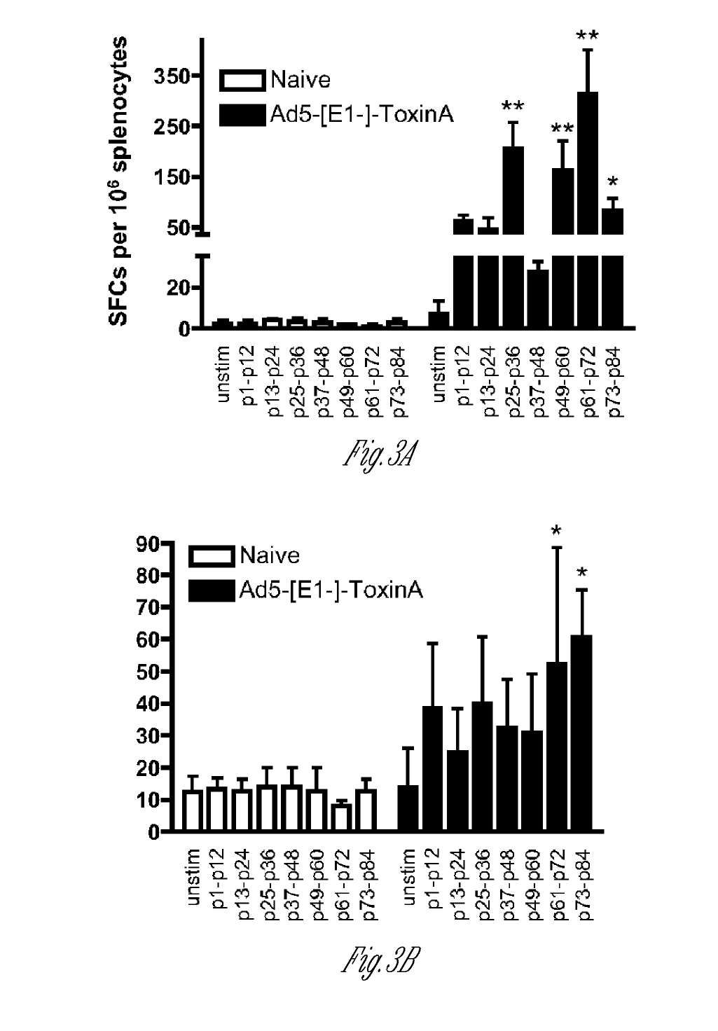

To examine if T cell responses to C. difficile Toxin A can be elicited by the potent Ad5-TA construct, cellular immune responses to peptides within the Ad5-TA construct were analyzed by ELISPOT assays. A 15-mer-peptide library was employed that spanned both N- and C-terminal portions of the C. difficile TA protein encoded by the Ad5-TA vaccine construct, where each peptide overlapped with its neighbor by 5 amino acids. Several clusters of immunogenic epitopes in the Toxin A non-enzymatic region were identified, with two being major immunodominant epitopes: VNGSRYYFDTDTAIA (SEQ ID NO:3) and YYFNTNTSIASTGYT (SEQ ID NO:4) (>400 IFNγ SFCs per 10⁶ splenocytes), (FIGS. 2A-2B) and Table 1). Further analysis utilizing an MHC I epitope prediction program (website at syfpeithi.de/scripts/MHCServer.dll/), indicates that two 9-mer peptides within the SEQ ID NO:3 and SEQ ID NO:4 peptides may bind well to the BALB/c H2K^{d} allele and be good MHC I epitopes. The sequences of these 9-mers are as follows.

(SEQ ID NO: 8)
YYFDTDTAI
and
(SEQ ID NO: 9)
YYFNTNTSI.

Administration of the Ad5-TA construct and analysis of splenocytes from immunized mice in an ELISPOT assay identified several other epitopes as also being highly immunogenic, including 3 epitopes yielding 150-400 SFCs, 4 yielding 100-150 SFCs and 2 epitopes yielding 50-100 SFCs per 10⁶ splenocytes in the IFNγ ELISpot (FIGS. 2A-2B, and Table 1).

TABLE 1
C. difficile toxin A-
Specific Major T cell Epitopes.

| | No. Spot | |
|---|---|---|
| Peptide | Forming Cells | SEQ ID NO: |
| #13 VNGSRYYFDTDTAIA | 400+ SFCs | SEQ ID NO: 3 |
| #63 YYFNTNTSIASTGYT | 400+ SFCs | SEQ ID NO: 4 |
| #9 NEKYYFNPNNAIAAV | 150-400 SFCs | SEQ ID NO: 10 |
| #51 QTIDGKKYYFNTNTF | 150-400 SFCs | SEQ ID NO: 11 |
| #55 VFKGPNGFEYFAPAN | 150-400 SFCs | SEQ ID NO: 12 |
| #27 YFNTNTAIASTGYTI | 100-150 SFCs | SEQ ID NO: 13 |
| #30 IGVFKGPNGFEYFAP | 100-150 SFCs | SEQ ID NO: 14 |
| #36 KYYFNPNNAIAAIHL | 100-150 SFCs | SEQ ID NO: 15 |
| #37 AAIHLCTINNDKYYF | 100-150 SFCs | SEQ ID NO: 16 |
| #66 QIGVFKGPDGFEYFA | 50-100 SFCs | SEQ ID NO: 17 |
| #74 NKNFYFRNGLPQIGV | 50-100 SFCs | SEQ ID NO: 18 |

The data in Table 1 were generated using an ELISPOT assay of splenocytes derived from Ad5-TA vaccinated BALB/c mice. The splenocytes were stimulated with 15-mer library spanning the C. difficile Toxin A region that is encoded by Ad-TA vaccine construct, followed by ELISPOT assay. In an EPISPOT assay, a peptide with high numbers of spot forming cells is an immunogenic epitope. Therefore, Table 1 provides the sequences of the peptides with the highest numbers of spot forming cells. The bold and underlined peptide sequences in peptides #13 and #63 were also identified by H2K^{d} (9-mers) epitope prediction (www.syfpeithi.de/scripts/MHCServer.dll/) as being good yields derivatives of peptides #13 and #63 as 2 top hits (highlighted in bold and underlined).

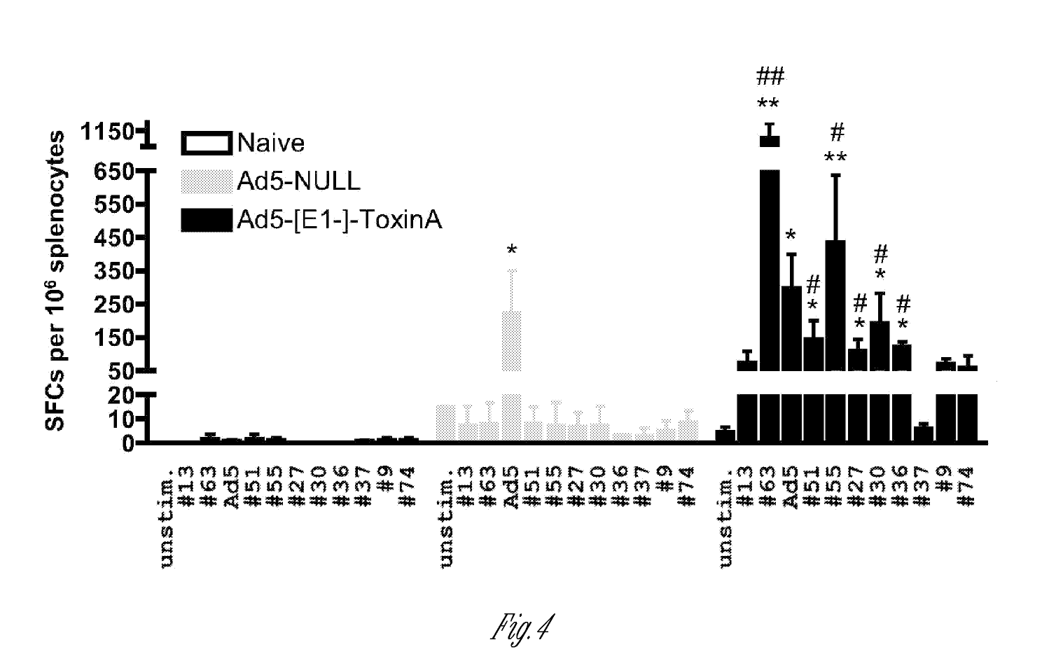

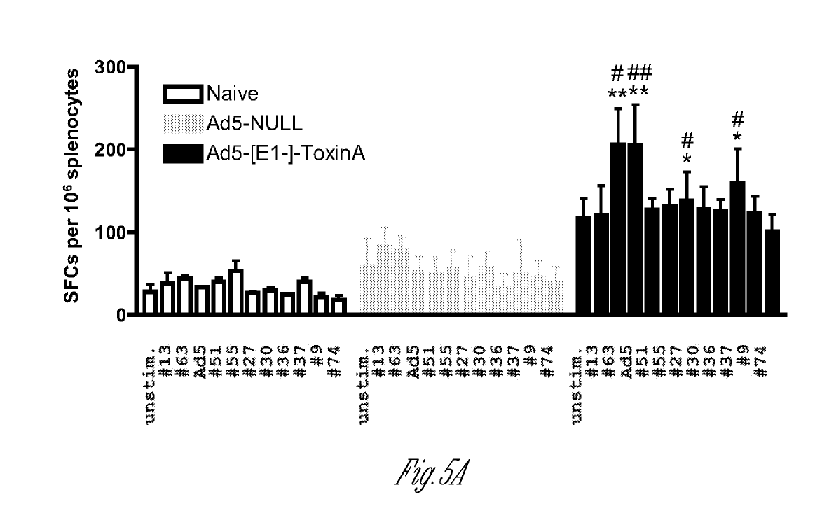

To further characterize T cell responses induced by the Ad5-TA C. difficile construct, splenocytes derived from Ad5-TA vaccinated (at 14 dpi) or naive mice were stimulated with peptide pools each containing twelve Toxin A-specific peptides. Utilizing these peptide pools, IFNγ or IL-2 based ELISPOT analyses were performed as well. As shown in FIG. 3A-3B, these IFNγ or IL-2 based ELISPOT analyses further confirmed that significant Toxin-A specific T cell responses were generated in Ad5-TA-vaccinated mice, but not in control mice. This study further demonstrates that use of the Ad5-TA construct induced a robust and broad T cell immune response to the C. difficile toxin A antigen, and that immunogenic T cell epitope clusters are located in several regions within the Toxin A C-terminal non-enzymatic domain, rather than being concentrated in just one particular region of the Toxin A molecule.

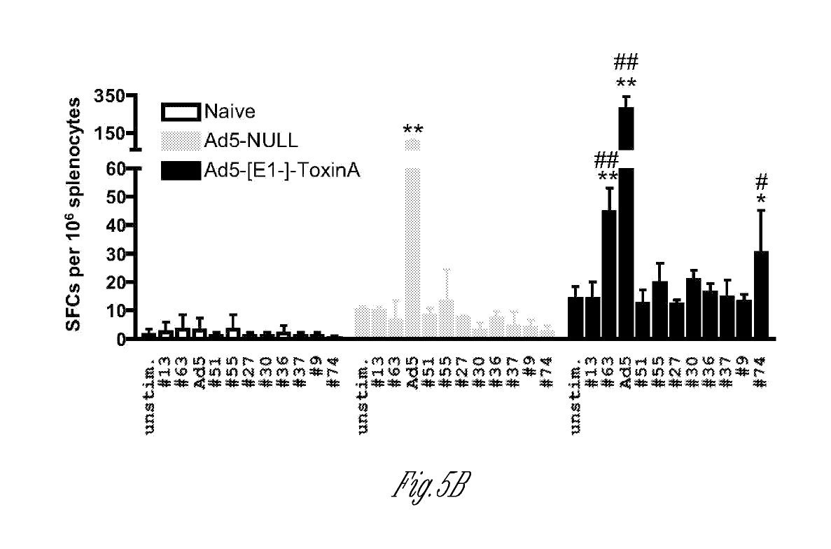

To assess the potential, non-specific Ad-mediated effects of vaccination, these responses were similarly analyzed after administration of an Ad5-Null control construct. An IFNγ ELISpot assay revealed a lack of any significant responses in both Ad-naïve and Ad5-Null injected mice, relative to the robust TA-specific T cell responses exhibited by Ad5-TA vaccinated mice (FIG. 4).

Significant Ad5-specific T cell responses were detected in Ad5-Null and Ad5-TA injected mice, which were identical between the two groups of Ad5-injected animals, indirectly confirming that both the Ad5 control and Ad5-TA vaccinations were performed identically and therefore C. difficile TA-specific T cell responses are truly derived only from the adenoviral-mediated expression of the Toxin A protein (FIG. 4).

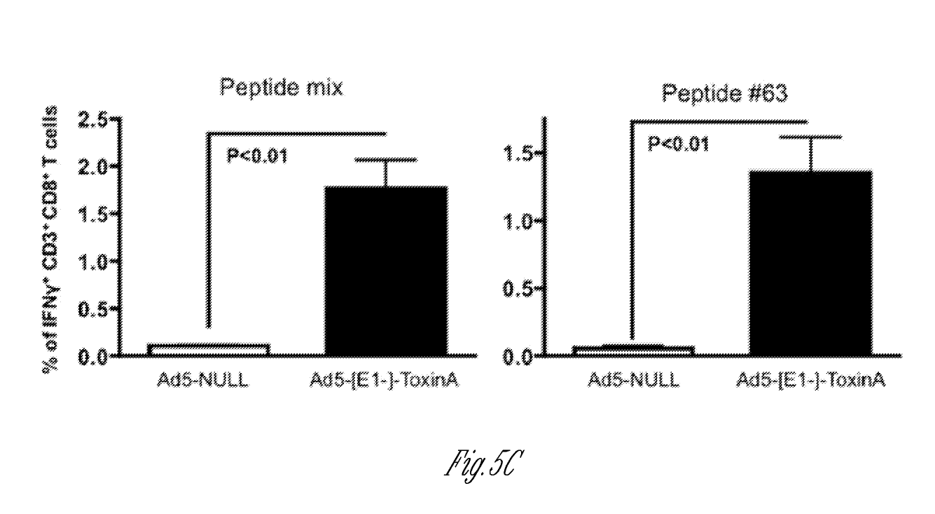

Ad5 vaccines typically induce CD8 specific effector T cell responses, however, CD4 responses can also be induced by Ad based vaccines (Appledorn et al., PLoS One 5(3):e9579 (2010); Seregin et al. Hum Gene Ther (April 2011), which are specifically incorporated by reference herein in their entireties. IL-4 and IL-2 Toxin A-specific ELISpot assays confirmed that a robust CD4 T cell response to Toxin A also occurred in Ad5-TA vaccinated mice. These data indicate that Ad5-TA administration induces robust pleiotropic CD8 and CD4 responses as well as potentially providing Th1/Th2 immunity to the C. difficile Toxin A protein (FIG. 5). The Ad5-TA vaccine is able to induce IFN′ production from CD8+ T cells in a recall response to TA-specific antigens (FIG. 5C).

Ad5-TA C. difficile Administration Completely Protects Mice from Lethal TA Challenge.

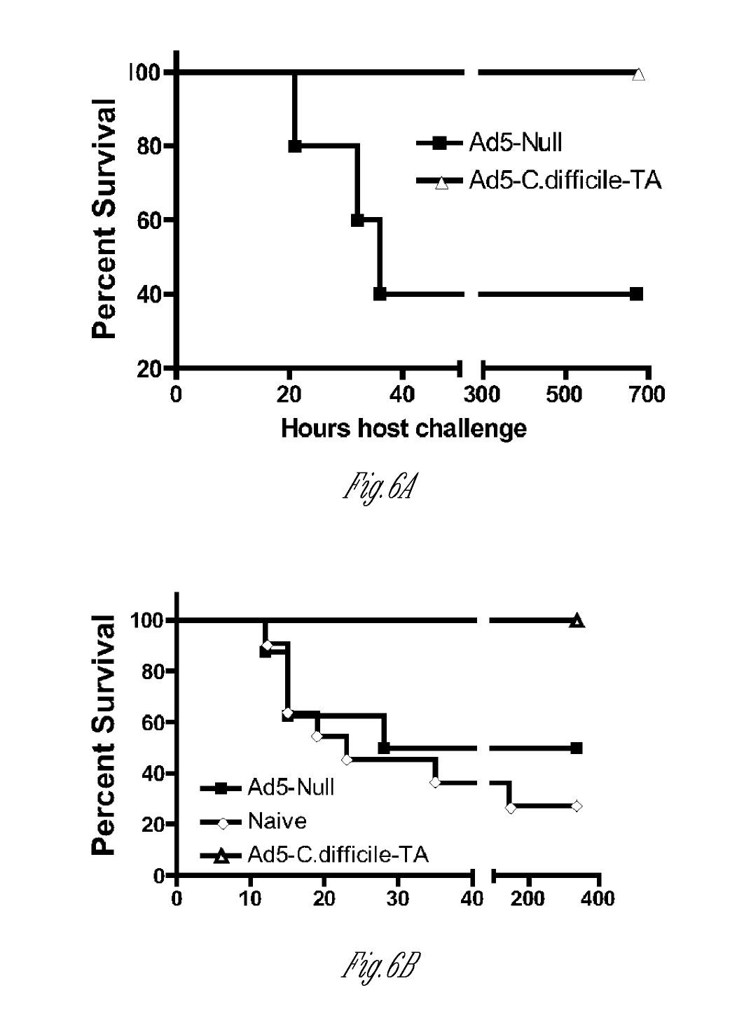

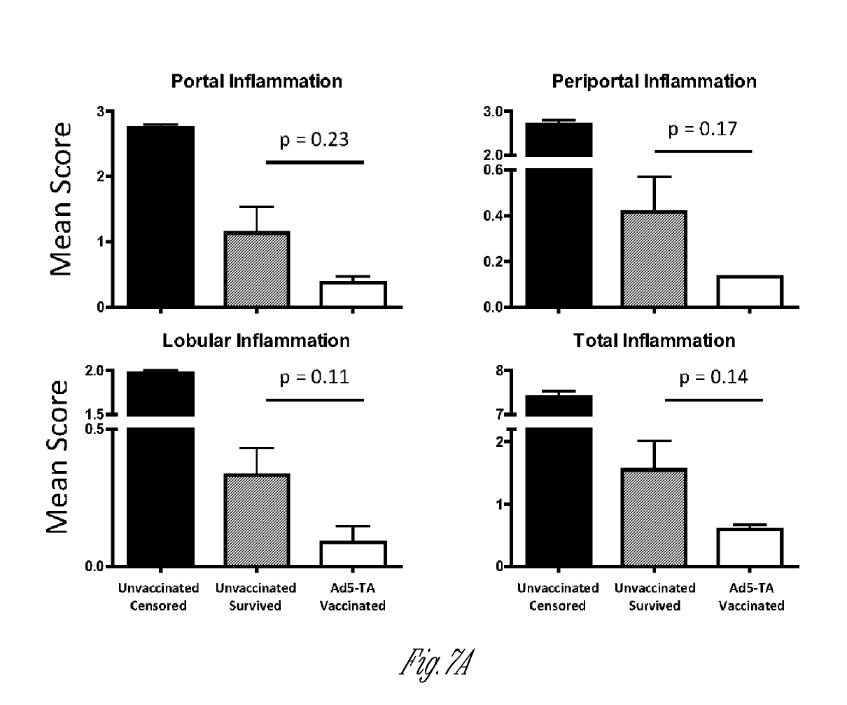

To determine if the rapid and robust induction of humoral and T cell responses elicited by vaccination with Ad-TA is clinically meaningful, mice were challenged with 300 ng (6×LD₅₀) of purified Toxin A 14 days after administration of the Ad5-TA construct. FIG. 6A shows that while the Ad5-Null injected mice have a 40% survival rate after this challenge, the Ad5-TA vaccinated mice have 100% survival for the duration of experiment. Thus, administration of the Ad5-TA construct significantly improved the survival of these animals (p<0.05) relative to use of the Ad5-Null control.

It was unclear, however, why some of Ad5-Null injected mice had survived the challenge, since similar challenges previously published resulted in 100% death of unvaccinated mice (Gardiner et al., Vaccine 27(27):3598-604 (2009)). The experiment was repeated using a Toxin A preparation from a source identical to the one used by Gardiner et al. (2009). Similar results to the first experiment were observed. While 100% of the Ad5-TA vaccinated mice survived Toxin A challenge, only about 50 percent of the Ad5-Null challenged mice survived while about 30% of naïve unvaccinated mice survived. Thus, the Ad5-TA vaccinated mice again exhibited significantly increased incidence of survival (p<0.05) as compared to unvaccinated mice (FIG. 6B).

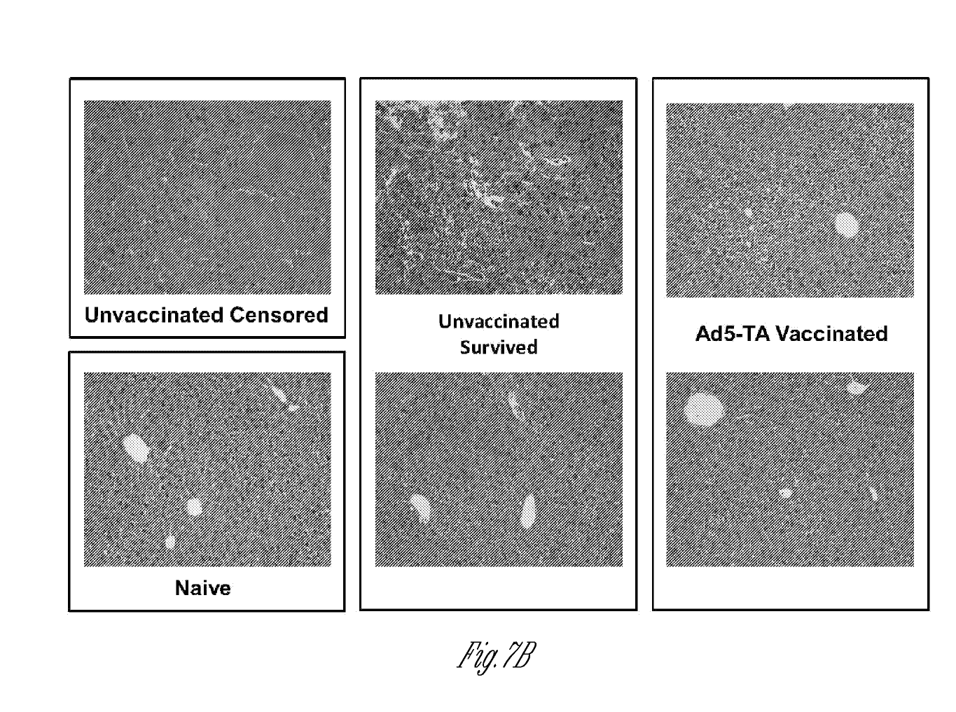

The severity of hepatic inflammation in Ad5-TA vaccinated mice after TA challenge was also significantly reduced. FIG. 7 shows that there was substantial portal, periportal and lobular hepatic inflammation in unvaccinated control mice that underwent Toxin A challenge. In contrast, the Ad5-TA vaccinated mice that were challenged with Toxin A exhibited reduced levels of portal, periportal and lobular hepatic inflammation. Significantly, the Ad5-TA vaccinated mice showed almost no signs of leukocyte invasion, lobular disarray, or necrosis. There was no apparent difference between the Ad5-TA group and the naïve mice. Unvaccinated Toxin A challenged mice had complete lobular disarray, with pooling of erythrocytes throughout the tissue subsequent to hemorrhage. The unvaccinated group of mice that survived this challenge had lobular disarray with focal areas of glassy eosinophilic hyalin deposition and necrosis. These mice also had areas of cellular swelling indicating cellular stress (FIG. 7B). Such damage was not seen in the Ad5-TA vaccinated mice. These data illustrate Ad5-TA vaccination reduces inflammation-related damage of tissues in mammals infected with Clostridium difficile.

Recurrence of Clostridium difficile associated diarrhea (CDAD) is associated with a lack of protective immunity to C. difficile toxins Toxin A and Toxin B. The incidence of CDAD reoccurrence ranges from 8 to 50% of cases with a trend to increase (Adam et al., The Lancet infectious diseases 5(9):549-57 (2005)). Patients developing high serum IgG antibodies titers against Toxin A during their first episode of CDAD are 48-fold less likely to develop recurrent CDAD (Ghose et al. Infect Immun 75(6):2826-32 (2007); Giannasca & Warny, Vaccine 22(7):848-56 (2004); Sougioultzis et al., Gastroenterology 128(3):764-70 (2005); Kyne et al., The New Eng J Med 342(6):390-7 (2000)).

The results described herein indicate that an immunological composition that includes the SEQ ID NO:2 antigen, or peptide antigens from within the SEQ ID NO:2 polypeptide has efficacy for treating and inhibiting infection of C. difficile. The dose regimen utilized in these studies (10¹⁰ viral particles/mouse) is a moderate adenoviral vector dose (Weaver et al., PLoS One 4(3):e5059 (2009); Gabitzsch et al., Vaccine (June 2009)). At least 20-fold higher doses have been utilized in mice for intramuscular immunizations (Pichla-Gollon et al., J Virol 83(11):5567-73 (2009)). Moreover, a dose of 10¹¹ viral particles/person is a currently FDA approved dose for Ad5-based vectors, and is a dose that has been utilized in clinical trials (see website at clinicaltrials.gov/ct2/show/NCT01147965). Even at these moderate dosages the Ad5-TA C. difficile construct disclosed here induced rapid (detectable as early as 3 days post-injection) and robust humoral and robust specific T cell responses to the C. difficile TA antigen. For example, T cell responses against the Ad5-TA construct were detectable as early as three days post injection.

The potency of this Ad5-TA C. difficile construct facilitated identification of important C. difficile TA specific immunogenic T cell epitopes, including those exhibited by the SEQ ID NO:3 and SEQ ID NO:4 peptides. Furthermore, the data described herein confirm that the Toxin A specific immune responses induced by the Ad5-TA construct positively correlate with full protection of mice against C. difficile Toxin A challenge, which typically causes significant mortality. Moreover, such protections against C. difficile Toxin A challenge was provided by the Ad5-TA construct, even when the challenge is performed as early as 14 days after a single, Ad5-TA vaccination, which is a time point much earlier than expected (see, e.g., Gardiner et al., Vaccine 27(27):3598-604 (2009)).

Therefore, an efficacious, Adenovirus based construct described herein can be used to treat and inhibit infection of C. difficile, as well as the side effects of C. difficile infection (e.g., inflammation-induced hepatic tissue damage). The Adenovirus based construct described herein encodes the C-terminal, highly immunogenic non-enzymatic region of the toxin A protein expressed by C. difficile (the SEQ ID NO:2 peptide). Even moderate doses of this construct result in rapid and robust inductions of both humoral and cellular C. difficile specific immune responses in mice. T cell responses were also observed and characterized. The data relating to T cell responses indicates that the clusters of immunogenic T cell epitopes described herein may contribute significantly to immunity against C. difficile.

### REFERENCES

[1] Pechine S, Janoir C, Collignon A. Variability of Clostridium difficile surface proteins and specific serum antibody response in patients with Clostridium difficile-associated disease. Journal of clinical microbiology 2005 October; 43(10):5018-25.
[2] Ghose C, Kalsy A, Sheikh A, Rollenhagen J, John M, Young J, et al. Transcutaneous immunization with Clostridium difficile toxoid A induces systemic and mucosal immune responses and toxin A-neutralizing antibodies in mice. Infect Immun 2007 June; 75(6):2826-32.
[3] Pechine S, Janoir C, Boureau H, Gleizes A, Tsapis N, Hoys S, et al. Diminished intestinal colonization by Clostridium difficile and immune response in mice after mucosal immunization with surface proteins of Clostridium difficile. Vaccine 2007 May 16; 25(20):3946-54.
[4] Aslam S, Hamill R J, Musher D M. Treatment of Clostridium difficile-associated disease: old therapies and new strategies. The Lancet infectious diseases 2005 September; 5(9):549-57.
[5] Hookman P, Barkin J S. Clostridium difficile associated infection, diarrhea and colitis. World J Gastroenterol 2009 Apr. 7; 15(13):1554-80.
[6] Gerding D N, Muto C A, Owens R C, Jr. Treatment of Clostridium difficile infection. Clin Infect Dis 2008 Jan. 15; 46 Suppl 1:S32-42.
[7] Giannasca P J, Warny M. Active and passive immunization against Clostridium difficile diarrhea and colitis. Vaccine 2004 Feb. 17; 22(7):848-56.
[8] Wright A, Drudy D, Kyne L, Brown K, Fairweather N F Immunoreactive cell wall proteins of Clostridium difficile identified by human sera. Journal of medical microbiology 2008 June; 57(Pt 6):750-6.
[9] Savidge T C, Pan W H, Newman P, O'Brien M, Anton P M, Pothoulakis C. Clostridium difficile toxin B is an inflammatory enterotoxin in human intestine. Gastroenterology 2003 August; 125(2):413-20.
[10] Hamm E E, Voth D E, Ballard J D. Identification of Clostridium difficile toxin B cardiotoxicity using a zebrafish embryo model of intoxication. Proc Natl Acad Sci USA 2006 Sep. 19; 103(38):14176-81.
[11] Johnson S, Kent S A, O'Leary K J, Merrigan M M, Sambol S P, Peterson L R, et al. Fatal pseudomembranous colitis associated with a variant clostridium difficile strain not detected by toxin A immunoassay. Annals of internal medicine 2001 Sep. 18; 135(6):434-8.
[12] Jacob S S, Sebastian J C, Hiorns D, Jacob S, Mukerjee P K. Clostridium difficile and acute respiratory distress syndrome. Heart Lung 2004 July-August; 33(4): 265-8.
[13] Sougioultzis S, Kyne L, Drudy D, Keates S, Maroo S, Pothoulakis C, et al. Clostridium difficile toxoid vaccine in recurrent C. difficile-associated diarrhea. Gastroenterology 2005 March; 128(3):764-70.
[14] Gardiner D F, Rosenberg T, Zaharatos J, Franco D, Ho D D. A DNA vaccine targeting the receptor-binding domain of Clostridium difficile toxin A. Vaccine 2009 Jun. 2; 27(27):3598-604.
[15] Castagliuolo I, Sardina M, Brun P, DeRos C, Mastrotto C, Lovato L, et al. Clostridium difficile toxin A carboxyl-terminus peptide lacking ADP-ribosyltransferase activity acts as a mucosal adjuvant. Infect Immun 2004 May; 72(5):2827-36.
[16] Brun P, Scarpa M, Grillo A, Palu G, Mengoli C, Zecconi A, et al. Clostridium difficile TxAC314 and SLP-36 kDa enhance the immune response toward a co-administered antigen. Journal of medical microbiology 2008 June; 57(Pt 6):725-31.
[17] Kotloff K L, Wasserman S S, Losonsky G A, Thomas W, Jr., Nichols R, Edelman R, et al. Safety and immunogenicity of increasing doses of a Clostridium difficile toxoid vaccine administered to healthy adults. Infect Immun 2001 February; 69(2):988-95.
[18] Aboudola S, Kotloff K L, Kyne L, Warny M, Kelly E C, Sougioultzis S, et al. Clostridium difficile vaccine and serum immunoglobulin G antibody response to toxin A. Infect Immun 2003 March; 71(3):1608-10.
[19] Kyne L, Warny M, Qamar A, Kelly C P. Asymptomatic carriage of Clostridium difficile and serum levels of IgG antibody against toxin A. The New England journal of medicine 2000 Feb. 10; 342(6):390-7.
[20] Kink J A, Williams J A. Antibodies to recombinant Clostridium difficile toxins A and B are an effective treatment and prevent relapse of C. difficile-associated disease in a hamster model of infection. Infect Immun 1998 May; 66(5):2018-25.
[21] Ward S J, Douce G, Figueiredo D, Dougan G, Wren B W. Immunogenicity of a Salmonella typhimurium aroA aroD vaccine expressing a nontoxic domain of Clostridium difficile toxin A. Infect Immun 1999 May; 67(5):2145-52.
[22] Appledorn D M, Aldhamen Y A, Depas W, Seregin S S, Liu C J, Schuldt N, et al. A new adenovirus based vaccine vector expressing an Eimeria tenella derived TLR agonist improves cellular immune responses to an antigenic target. PLoS One 2010; 5(3):e9579.
[23] Seregin S S, Aldhamen Y A, Appledorn D M, Zehnder J, Voss T, Godbehere S, et al. Use of DAF-Displaying Adenovirus Vectors Reduces Induction of Transgene- and Vector-Specific Adaptive Immune Responses in Mice. Hum Gene Ther 2011 Apr. 18.
[24] Chen X, Katchar K, Goldsmith J D, Nanthakumar N, Cheknis A, Gerding D N, et al. A mouse model of Clostridium difficile-associated disease. Gastroenterology 2008 December; 135(6): 1984-92.
[25] Taylor C P, Tummala S, Molrine D, Davidson L, Farrell R J, Lembo A, et al. Open-label, dose escalation phase I study in healthy volunteers to evaluate the safety and pharmacokinetics of a human monoclonal antibody to Clostridium difficile toxin A. Vaccine 2008 Jun. 25; 26(27-28):3404-9.
[26] Seregin S S, Amalfitano A. Gene Therapy for Lysosomal Storage Diseases: Progress, Challenges and Future Prospects. Current Pharmaceutical Design 2011; in press.
[27] Wang R, Doolan D L, Le T P, Hedstrom R C, Coonan K M, Charoenvit Y, et al. Induction of antigen-specific cytotoxic T lymphocytes in humans by a malaria DNA vaccine. Science 1998 Oct. 16; 282(5388):476-80.
[28] Asmuth D M, Brown E L, Dinubile M J, Sun X, Del Rio C, Harro C, et al. Comparative Cell-Mediated Immunogenicity of DNA/DNA, DNA/Adenovirus Type 5 (Ad5), or Ad5/Ad5 HIV-1 Clade B gag Vaccine Prime-Boost Regimens. J Infect Dis 2010 Jan. 1; 201(1):132-41.
[29] Liu J, O'Brien K L, Lynch D M, Simmons N L, La Porte A, Riggs A M, et al Immune control of an SIV challenge by a T-cell-based vaccine in rhesus monkeys. Nature 2009 Jan. 1; 457(7225):87-91.
[30] Shott J P, McGrath S M, Pau M G, Custers J H, Ophorst 0, Demoitie M A, et al. Adenovirus 5 and 35 vectors expressing Plasmodium falciparum circumsporozoite surface protein elicit potent antigen-specific cellular IFN-gamma and antibody responses in mice. Vaccine 2008 Jun. 2; 26(23):2818-23.
[31] Abbink P, Lemckert A A, Ewald B A, Lynch D M, Denholtz M, Smits S, et al. Comparative seroprevalence and immunogenicity of six rare serotype recombinant adenovirus vaccine vectors from subgroups B and D. J Virol 2007 May; 81(9):4654-63.
[32] Barouch D H, Pau M G, Custers J H, Koudstaal W, Kostense S, Havenga M J, et al Immunogenicity of recombinant adenovirus serotype 35 vaccine in the presence of pre-existing anti-Ad5 immunity. J Immunol 2004 May 15; 172(10):6290-7.
[33] Allaker R P. Host defence peptides—a bridge between the innate and adaptive immune responses. Transactions of the Royal Society of Tropical Medicine and Hygiene 2008 January; 102(1):3-4.
[34] Iwasaki A, Medzhitov R. Toll-like receptor control of the adaptive immune responses. Nature immunology 2004 October; S(10):987-95.
[35] Kemper C, Atkinson J P. T-cell regulation: with complements from innate immunity. Nat Rev Immunol 2007 January; 7(1):9-18.
[36] Morgan B P, Marchbank K J, Longhi M P, Harris C L, Gallimore A M. Complement: central to innate immunity and bridging to adaptive responses. Immunol Lett 2005 Mar. 15; 97(2):171-9.
[37] Catanzaro A T, Koup R A, Roederer M, Bailer R T, Enama M E, Moodie Z, et al. Phase 1 safety and immunogenicity evaluation of a multiclade HIV-1 candidate vaccine delivered by a replication-defective recombinant adenovirus vector. J Infect Dis 2006 Dec. 15; 194(12):1638-49.
[38] Gao W, Soloff A C, Lu X, Montecalvo A, Nguyen D C, Matsuoka Y, et al. Protection of mice and poultry from lethal H5N1 avian influenza virus through adenovirus-based immunization. J Virol 2006 February; 80(4):1959-64.
[39] Sullivan N J, Sanchez A, Rollin P E, Yang Z Y, Nabel G J. Development of a preventive vaccine for Ebola virus infection in primates. Nature 2000 Nov. 30; 408(6812):605-9.
[40] Pinto A R, Fitzgerald J C, Giles-Davis W, Gao G P, Wilson J M, Ertl H C. Induction of C D8+ T cells to an HIV-1 antigen through a prime boost regimen with heterologous E1-deleted adenoviral vaccine carriers. J Immunol 2003 Dec. 15; 171(12):6774-9.
[41] Reyes-Sandoval A, Sridhar S, Berthoud T, Moore A C, Harty J T, Gilbert S C, et al. Single-dose immunogenicity and protective efficacy of simian adenoviral vectors against Plasmodium berghei. Eur J Immunol 2008 March; 38(3):732-41.
[42] Shiver J W, Fu T M, Chen L, Casimiro D R, Davies M E, Evans R K, et al. Replication-incompetent adenoviral vaccine vector elicits effective anti-immunodeficiency-virus immunity. Nature 2002 Jan. 17; 415(6869):331-5.
[43] Schirmbeck R, Reimann J, Kochanek S, Kreppel F. The immunogenicity of adenovirus vectors limits the multispecificity of C D8 T-cell responses to vector-encoded transgenic antigens. Mol Ther 2008 September; 16(9):1609-16.
[44] Buchbinder S P, Mehrotra D V, Duerr A, Fitzgerald D W, Mogg R, Li D, et al. Efficacy assessment of a cell-mediated immunity HIV-1 vaccine (the Step Study): a double-blind, randomised, placebo-controlled, test-of-concept trial. Lancet 2008 Nov. 29; 372(9653):1881-93.
[45] Weaver E A, Nehete P N, Buchl S S, Senac J S, Palmer D, Ng P, et al. Comparison of replication-competent, first generation, and helper-dependent adenoviral vaccines. PLoS One 2009; 4(3):e5059.
[46] Gabitzsch E S, Xu Y, Yoshida L H, Balint J, Amalfitano A, Jones F R. Novel Adenovirus type 5 vaccine platform induces cellular immunity against HIV-1 Gag, Pol, Nef despite the presence of Ad5 immunity. Vaccine 2009 Jun. 24.
[47] Pichla-Gollon S L, Lin S W, Hensley S E, Lasaro M O, Herkenhoff-Haut L, Drinker M, et al. Effect of preexisting immunity on an adenovirus vaccine vector: in vitro neutralization assays fail to predict inhibition by antiviral antibody in vivo. J Virol 2009 June; 83(11):5567-73.
[48] Seregin S S, Aldhamen Y A, Appledorn D M, Hartman Z C, Schuldt N J, Scott J, et al. Adenovirus capsid-display of the retro-oriented human complement inhibitor DAF reduces Ad vector-triggered immune responses in vitro and in vivo. Blood 2010 Sep. 9; 116(10):1669-77.
[49] Ahearn J M, Fischer M B, Croix D, Goerg S, Ma M, Xia J, et al. Disruption of the Cr2 locus results in a reduction in B-1a cells and in an impaired B cell response to T-dependent antigen. Immunity 1996 March; 4(3):251-62.
[50] Ng P, Graham F L. Construction of first-generation adenoviral vectors. Methods in molecular medicine 2002; 69:389-414.
[51] Seregin S S, Aldhamen Y A, Appledorn D M, Schuldt N J, McBride A J, Bujold M, et al. CR1/2 is an important suppressor of Adenovirus-induced innate immune responses and is required for induction of neutralizing antibodies. Gene Ther 2009 October; 16(10):1245-59.
[52] Seregin S S, Appledorn D M, McBride A J, Schuldt N J, Aldhamen Y A, Voss T, et al. Transient pretreatment with glucocorticoid ablates innate toxicity of systemically delivered adenoviral vectors without reducing efficacy. Mol Ther 2009 April; 17(4):685-96.
[53] Hensley S E, Cun A S, Giles-Davis W, Li Y, Xiang Z, Lasaro M O, et al. Type I interferon inhibits antibody responses induced by a chimpanzee adenovirus vector. Mol Ther 2007 February; 15(2):393-403.
[54] Weaver E A, Barry M A. Effects of Shielding Adenoviral Vectors with Polyethylene Glycol (PEG) on Vector-specific and Vaccine-mediated Immune Responses. Hum Gene Ther 2008 Sep. 8.
[55] Hu H, Serra D, Amalfitano A. Persistence of an [E1-, polymerase-] adenovirus vector despite transduction of a neoantigen into immune-competent mice. Hum Gene Ther 1999 Feb. 10; 10(3):355-64.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The specific methods, devices and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a bioreactor” or “a nucleic acid” or “a polypeptide” includes a plurality of such bioreactors, nucleic acids or polypeptides (for example, a solution of nucleic acids or polypeptides or a series of nucleic acid or polypeptide preparations), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

The following statements of the invention are intended to describe certain features of the invention.

### STATEMENTS OF THE INVENTION

1. A peptide antigen with an amino acid sequence comprising at least 15 contiguous amino acids of SEQ ID NO:2, and/or with an amino acid sequence comprising 95% sequence identity to SEQ ID NO:2.
2. The peptide antigen of statement 1, with an amino acid sequence comprising 95% sequence identity to any of SEQ ID NO:2, SEQ ID NO:3, SEQ ID NO:4, SEQ ID NO:7, SEQ ID NO:8, SEQ ID NO:9, SEQ ID NO:10, SEQ ID NO:11, SEQ ID NO:12, SEQ ID NO:13, SEQ ID NO:14 SEQ ID NO:15, SEQ ID NO:16, SEQ ID NO:17, or SEQ ID NO:18.
3. The peptide antigen of statement 1 or 2, with an amino acid sequence consisting essentially of SEQ ID NO:2, SEQ ID NO:3, SEQ ID NO:4, SEQ ID NO:7, SEQ ID NO:8, SEQ ID NO:9, SEQ ID NO:10, SEQ ID NO:11, SEQ ID NO:12, SEQ ID NO:13, SEQ ID NO:14 SEQ ID NO:15, SEQ ID NO:16, SEQ ID NO:17, SEQ ID NO:18, or a combination thereof
4. The peptide antigen of statement 1, 2 or 3, further comprising a toxin B peptide antigen.
5. The peptide antigen of statement 4, wherein the toxin B peptide antigen is a peptide comprising at least 15 contiguous amino acids of SEQ ID NO:6 or SEQ ID NO:7 and/or has an amino acid sequence comprising
95% sequence identity to SEQ ID NO:6 or SEQ ID NO:7.
6. The peptide antigen of statement 4, wherein the toxin B peptide antigen consists essentially of SEQ ID NO:7.
7. The peptide antigen of any of statements 4-6, wherein the SEQ ID NO:2 peptide antigen and the toxin B antigen are separate peptide antigens.
8. The peptide antigen of any of statements 4-6, wherein the SEQ ID NO:2 peptide antigen and the toxin B antigen are linked together.
9. A composition comprising an effective amount of the peptide antigen of any of statements 1-9.
10. An expression cassette comprising a nucleic acid encoding any of the peptide antigens of any of statements 1-9 operably linked to transcriptional regulatory element.
11. An expression cassette comprising a nucleic acid encoding a combination of any the peptide antigens of any of statements 1-9 operably linked to transcriptional regulatory element.
12. The expression cassette of statement 10 or 11, wherein the transcriptional regulatory element is selected from the group consisting of a promoter, an enhancer, a terminator of transcription, or a combination thereof
13. A composition comprising an effective amount of the expression cassette of any of statements 10-12.
14. A vector comprising the expression cassette of any of statements 10-12.
15. The vector of statement 14, wherein the vector is a viral vector.
16. The vector of statement 14 or 15, wherein the vector is replication incompetent viral vector.
17. The vector of any of statements 14-16, wherein the vector is an adenoviral vector.
18. A composition comprising an effective amount of the vector of any of statements 14-17.
19. A method of treating or inhibiting infection of Clostridium difficile in a mammal comprising administering the composition of any of statements 9, 13 or 18 to the mammal.
20. The method of statement 19, wherein the mammal is a human.
21. The method of statement 19 or 20, further comprising administering the composition a second time.
22. The method of any of statements 19-21, wherein inflammation-related damage of tissues is reduced the mammal relative to a mammal who did not receive administration of the composition.
23. Use of the composition of any of statements 9, 13 or 18 for treating or inhibiting infection of Clostridium difficile in a mammal
24. Use of an immunological composition comprising an effective amount of a replication incompetent adenoviral vector adapted to express a Clostridium difficile toxin A peptide having SEQ ID NO:2, or a peptide with an amino acid sequence comprising 95% sequence identity to SEQ ID NO:2 for treating or inhibiting infection of Clostridium difficile in a mammal.

Other embodiments are described within the following claims.

1. An immunological composition comprising an effective amount of:
(a) a Clostridium difficile toxin A peptide with a sequence selected from the group consisting of SEQ ID NO:2, SEQ ID NO:3, SEQ ID NO:4, a sequence comprising 95% sequence identity to SEQ ID NO:2, or a combination thereof;
(b) an expression vector adapted to express the Clostridium difficile toxin A peptide; or
(c) a combination thereof.

2. The composition of claim 1, wherein the expression vector is a recombinant adenovirus, retrovirus, lentivirus, herpesvirus, poxvirus, papilloma virus, or adeno-associated virus.

3. The composition of claim 1, wherein the expression vector is a replication incompetent adenoviral vector.

4. The composition of claim 1, wherein the composition further comprises an effective amount of a Clostridium difficile toxin B peptide having SEQ ID NO:7; a second replication incompetent adenoviral vector that is adapted to express a Clostridium difficile toxin B peptide having SEQ ID NO:7; or a combination thereof.

5. The composition of claim 3, wherein the adenoviral vector and/or the second adenoviral vector is a serotype 5 human adenovirus.

6. A method of treating or inhibiting infection of Clostridium difficile in a mammal comprising administering a composition comprising:
(a) a Clostridium difficile toxin A peptide with a sequence selected from the group consisting of SEQ ID NO:2, SEQ ID NO:3, SEQ ID NO:4, a sequence comprising 95% sequence identity to SEQ ID NO:2, or a combination thereof;
(b) expression vector adapted to express the Clostridium difficile toxin A peptide; or
(c) a combination thereof.

7. The method of claim 6, wherein the expression vector is a recombinant adenovirus, retrovirus, lentivirus, herpesvirus, poxvirus, papilloma virus, or adeno-associated virus.

8. The method of claim 6, wherein the expression vector is a replication incompetent adenoviral vector.

9. The method of claim 6, wherein the composition further comprises an effective amount of a Clostridium difficile toxin B peptide comprising SEQ ID NO:7; a second replication incompetent adenoviral vector that is adapted to express a Clostridium difficile toxin B peptide comprising SEQ ID NO:7; or a combination thereof.

10. The method of claim 8, wherein the adenoviral vector and/or the second adenoviral vector is a serotype 5 human adenovirus.

11. The method of claim 6, further comprising administering the composition a second or a third time.

12. A peptide antigen with an amino acid sequence comprising at least 15 contiguous amino acids of SEQ ID NO:2, and/or with an amino acid sequence comprising 95% sequence identity to SEQ ID NO:2.

13. The peptide antigen of claim 12, with an amino acid sequence comprising 95% sequence identity to any of SEQ ID NO:2, SEQ ID NO:3, SEQ ID NO:4, SEQ ID NO:7, SEQ ID NO:8, SEQ ID NO:9, SEQ ID NO:10, SEQ ID NO:11, SEQ ID NO:12, SEQ ID NO:13, SEQ ID NO:14 SEQ ID NO:15, SEQ ID NO:16, SEQ ID NO:17, SEQ ID NO:18, or a combination thereof.

14. An expression cassette comprising a nucleic acid encoding the peptide antigen of claim 12 operably linked to transcriptional regulatory element.

15. The expression cassette of claim 14, wherein the transcriptional regulatory element is selected from the group consisting of a promoter, an enhancer, a terminator of transcription, or a combination thereof.

16. A vector comprising the expression cassette of claim 14.

17. The vector of claim 16, wherein the vector comprises a recombinant adenovirus, retrovirus, lentivirus, herpesvirus, poxvirus, papilloma virus, or adeno-associated virus.

18. The vector of claim 16, wherein the vector is replication incompetent viral vector.

19. The vector of claim 16, wherein the vector is an adenoviral vector.

20. A composition comprising an effective amount of the vector of claim 16.

21. An expression cassette comprising a nucleic acid encoding the peptide antigen of claim 13 operably linked to transcriptional regulatory element.


SEQUENCE LISTING (18 sequences)

SEQ ID NO: 1 - protein, 2710 aa, C. difficile
MSLISKEELIKLAYSIRPRENEYKTILTNLDEYNKLTTNNNENKYLQLKKLNESIDVFMNKYKTSSRNRALSNLKKDILKEVILIKNSNTSPVEKNLHFVWIGGEVSDIALEYIKQWADINAEYNIKLWYDSEAFLVNTLKKAIVESSTTEALQLLEEEIQNPQFDNMKFYKKRMEFIYDRQKRFINYYKSQINKPTVPTIDDIIKSHLVSEYNRDETVLESYRTNSLRKINSNHGIDIRANSLFTEQELLNIYSQELLNRGNLAAASDIVRLLALKNFGGVYLDVDMLPGIHSDLFKTISRPSSIGLDRWEMIKLEAIMKYKKYINNYTSENFDKLDQQLKDNFKLIIESKSEKSEIFSKLENLNVSDLEIKIAFALGSVINQALISKQGSYLTNLVIEQVKNRYQFLNQHLNPAIESDNNFTDTTKIFHDSLFNSATAENSMFLTKIAPYLQVGFMPEARSTISLSGPGAYASAYYDFINLQENTIEKTLKASDLIEFKFPENNLSQLTEQEINSLWSFDQASAKYQFEKYVRDYTGGSLSEDNGVDFNKNTALDKNYLLNNKIPSNNVEEAGSKNYVHYIIQLQGDDISYEATCNLFSKNPKNSIIIQRNMNESAKSYFLSDDGESILELNKYRIPERLKNKEKVKVTFIGHGKDEFNTSEFARLSVDSLSNEISSFLDTIKLDISPKNVEVNLLGCNMFSYDFNVEETYPGKLLLSIMDKITSTLPDVNKNSITIGANQYEVRINSEGRKELLAHSGKWINKEEAIMSDLSSKEYIFFDSIDNKLKAKSKNIPGLASISEDIKTLLLDASVSPDTKFILNNLKLNIESSIGDYIYYEKLEPVKNIIHNSIDDLIDEFNLLENVSDELYELKKLNNLDEKYLISFEDISKNNSTYSVRFINKSNGESVYVETEKEIFSKYSEHITKEISTIKNSIITDVNGNLLDNIQLDHTSQVNTLNAAFFIQSLIDYSSNKDVLNDLSTSVKVQLYAQLFSTGLNTIYDSIQLVNLISNAVNDTINVLPTITEGIPIVSTILDGINLGAAIKELLDEHDPLLKKELEAKVGVLAINMSLSIAATVASIVGIGAEVTIFLLPIAGISAGIPSLVNNELILHDKATSVVNYFNHLSESKKYGPLKTEDDKILVPIDDLVISEIDFNNNSIKLGTCNILAMEGGSGHTVTGNIDHFFSSPSISSHIPSLSIYSAIGIETENLDFSKKIMMLPNAPSRVFWWETGAVPGLRSLENDGTRLLDSIRDLYPGKFYWRFYAFFDYAITTLKPVYEDTNIKIKLDKDTRNFIMPTITTNEIRNKLSYSFDGAGGTYSLLLSSYPISTNINLSKDDLWIFNIDNEVREISIENGTIKKGKLIKDVLSKIDINKNKLIIGNQTIDFSGDIDNKDRYIFLTCELDDKISLIIEINLVAKSYSLLLSGDKNYLISNLSNTIEKINTLGLDSKNIAYNYTDESNNKYFGAISKTSQKSIIHYKKDSKNILEFYNDSTLEFNSKDFIAEDINVFMKDDINTITGKYYVDNNTDKSIDFSISLVSKNQVKVNGLYLNESVYSSYLDFVKNSDGHHNTSNFMNLFLDNISFWKLFGFENINFVIDKYFTLVGKTNLGYVEFICDNNKNIDIYFGEWKTSSSKSTIFSGNGRNVVVEPIYNPDTGEDISTSLDFSYEPLYGIDRYINKVLIAPDLYTSLININTNYYSNEYYPEIIVLNPNTFHKKVNINLDSSSFEYKWSTEGSDFILVRYLEESNKKILQKIRIKGILSNTQSFNKMSIDFKDIKKLSLGYIMSNFKSFNSENELDRDHLGFKIIDNKTYYYDEDSKLVKGLININNSLFYFDPIEFNLVTGWQTINGKKYYFDINTGAALTSYKIINGKHFYFNNDGVMQLGVFKGPDGFEYFAPANTQNNNIEGQAIVYQSKFLTLNGKKYYFDNNSKAVTGWRIINNEKYYFNPNNAIAAVGLQVIDNNKYYFNPDTAIISKGWQTVNGSRYYFDTDTAIAFNGYKTIDGKHFYFDSDCVVKIGVFSTSNGFEYFAPANTYNNNIEGQAIVYQSKFLTLNGKKYYFDNNSKAVTGLQTIDSKKYYFNTNTAEAATGWQTIDGKKYYFNTNTAEAATGWQTIDGKKYYFNTNTAIASTGYTIINGKHFYFNTDGIMQIGVFKGPNGFEYFAPANTDANNIEGQAILYQNEFLTLNGKKYYFGSDSKAVTGWRIINNKKYYFNPNNAIAAIHLCTINNDKYYFSYDGILQNGYITIERNNFYFDANNESKMVTGVFKGPNGFEYFAPANTHNNNIEGQAIVYQNKFLTLNGKKYYFDNDSKAVTGWQTIDGKKYYFNLNTAEAATGWQTIDGKKYYFNLNTAEAATGWQTIDGKKYYFNTNTFIASTGYTSINGKHFYFNTDGIMQIGVFKGPNGFEYFAPANTDANNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDTAMAAAGGLFEIDGVIYFFGVDGVKAPGIYG

SEQ ID NO: 2 - protein, 810 aa, C. difficile
ALTSYKIINGKHFYFNNDGVMQLGVFKGPDGFEYFAPANTQNNNIEGQAIVYQSKFLTLNGKKYYFDNNSKAVTGWRIINNEKYYFNPNNAIAAVGLQVIDNNKYYFNPDTAIISKGWQTVNGSRYYFDTDTAIAFNGYKTIDGKHFYFDSDCVVKIGVFSTSNGFEYFAPANTYNNNIEGQAIVYQSKFLTLNGKKYYFDNNSKAVTGLQTIDSKKYYFNTNTAEAATGWQTIDGKKYYFNTNTAEAATGWQTIDGKKYYFNTNTAIASTGYTIINGKHFYFNTDGIMQIGVFKGPNGFEYFAPANTDANNIEGQAILYQNEFLTLNGKKYYFGSDSKAVTGWRIINNKKYYFNPNNAIAAIHLCTINNDKYYFSYDGILQNGYITIERNNFYFDANNESKMVTGVFKGPNGFEYFAPANTHNNNIEGQAIVYQNKFLTLNGKKYYFDNDSKAVTGWQTIDGKKYYFNLNTAEAATGWQTIDGKKYYFNLNTAEAATGWQTIDGKKYYFNTNTFIASTGYTSINGKHFYFNTDGIMQIGVFKGPNGFEYFAPANTDANNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDT

SEQ ID NO: 3 - protein, 15 aa, C. difficile
VNGSRYYFDTDTAIA

SEQ ID NO: 4 - protein, 15 aa, C. difficile
YYFNTNTSIASTGYT

SEQ ID NO: 5 - DNA, 9770 bp, C. difficile
aaagtgttctatctaatatgaagatttaccaataaaaaggtggactatgatgaatgcacagtagttcacctttttatatttctaatggtaacaaaatatttttttatataaacctaggaggcgttatgaatatgacaatatcttttttatcagagcatatatttataaagttagtaattttaactatatcatttgatacattattaggatgtttaagtgcaataaaaagtcgtaaatttaattctagttttggaatagatggaggaatcagaaaagtagcaatgatagcatgtatattttttttatcagtagttgacattcttacaaagtttaactttttatttatgttaccacaagattgtatcaattttttaagactaaaacatcttggaatatctgaatttttctctattttatttattttatatgaaagtgtaagtatattaaaaaatatgtgcttatgtggattaccagtacctaagagattaaaggaaaaaatagcaattttactagatgcaatgacagatgaaatgaatgctaaggatgaaaagtaagtaatggtagatataataaagatattaacaaataaaaagtgttatccaaataagaatagctgaaagttatcataattcatgaaactaataatgaaaacgagggagcagatgccaagagacacacaagtattaaatacatataatttcgaagcaagtgttcattactatatagatgacaaggtagtatatcaaacattggttcacaaagatggtgcatggtcagttggtaaaatctattaagctacattagttacagatatcacaaactataatagttaaacatagaaatatgtgtaaattgtgatggaaattattcaaaaacacaaaaatacgtgatgaaggacaaaatgatatagaaaataagtatcaaaccttaataaatgatttaattgatagtttaaaagttataggaaaaatatataaagaaataaaaacattaaaaaaatataagatatgtttacaaattactatcagacaatctccttatctaatagaagagtcaattaactaattgagtatctttaaattgaaatgttaggaagtgatttaaatatgaaaacttaaattataaaaaatcaatattaatttatttttaaaaaatagaaaggagtgtataagatttattttcaaagtttaaaaacaagaaaatcaatttaaatttcagaaggaataaatgtggttatagaagtggatttattatcaaaaataataatactaggaggtttttatgtctttaatatctaaagaagagttaataaaactcgcatatagcattagaccaagagaaaatgagtataaaactatactaactaatttagacgaatataataagttaactacaaacaataatgaaaataaatatttgcaattaaaaaaactaaatgaatcaattgatgtttttatgaataaatataaaacttcaagcagaaatagagcactctctaatctaaaaaaagatatattaaaagaagtaattcttattaaaaattccaatacaagccctgtagaaaaaaatttacattttgtatggataggtggagaagtcagtgatattgctcttgaatacataaaacaatgggctgatattaatgcagaatataatattaaactgtggtatgatagtgaagcattcttagtaaatacactaaaaaaggctatagttgaatcttctaccactgaagcattacagctactagaggaagagattcaaaatcctcaatttgataatatgaaattttacaaaaaaaggatggaatttatatatgatagacaaaaaaggtttataaattattataaatctcaaatcaataaacctacagtacctacaatagatgatattataaagtctcatctagtatctgaatataatagagatgaaactgtattagaatcatatagaacaaattctttgagaaaaataaatagtaatcatgggatagatatcagggctaatagtttgtttacagaacaagagttattaaatatttatagtcaggagttgttaaatcgtggaaatttagctgcagcatctgacatagtaagattattagccctaaaaaattttggcggagtatatttagatgttgatatgcttccaggtattcactctgatttatttaaaacaatatctagacctagctctattggactagaccgttgggaaatgataaaattagaggctattatgaagtataaaaaatatataaataattatacatcagaaaactttgataaacttgatcaacaattaaaagataattttaaactcattatagaaagtaaaagtgaaaaatctgagatattttctaaattagaaaatttaaatgtatctgatcttgaaattaaaatagctttcgctttaggcagtgttataaatcaagccttgatatcaaaacaaggttcatatcttactaacctagtaatagaacaagtaaaaaatagatatcaatttttaaaccaacaccttaacccagccatagagtctgataataacttcacagatactactaaaatttttcatgattcattatttaattcagctaccgcagaaaactctatgtttttaacaaaaatagcaccatacttacaagtaggttttatgccagaagctcgctccacaataagtttaagtggtccaggagcttatgcgtcagcttactatgatttcataaatttacaagaaaatactatagaaaaaactttaaaagcatcagatttaatagaatttaaattcccagaaaataatctatctcaattgacagaacaagaaataaatagtctatggagctttgatcaagcaagtgcaaaatatcaatttgagaaatatgtaagagattatactggtggatctctttctgaagacaatggggtagactttaataaaaatactgccctcgacaaaaactatttattaaataataaaattccatcaaacaatgtagaagaagctggaagtaaaaattatgttcattatatcatacagttacaaggagatgatataagttatgaagcaacatgcaatttattttctaaaaatcctaaaaatagtattattatacaacgaaatatgaatgaaagtgcaaaaagctactttttaagtgatgatggagaatctattttagaattaaataaatataggatacctgaaagattaaaaaataaggaaaaagtaaaagtaacctttattggacatggtaaagatgaattcaacacaagcgaatttgctagattaagtgtagattcactttccaatgagataagttcatttttagataccataaaattagatatatcacctaaaaatgtagaagtaaacttacttggatgtaatatgtttagttatgattttaatgttgaagaaacttatcctgggaagttgctattaagtattatggacaaaattacttccactttacctgatgtaaataaaaattctattactataggagcaaatcaatatgaagtaagaattaatagtgagggaagaaaagaacttctggctcactcaggtaaatggataaataaagaagaagctattatgagcgatttatctagtaaagaatacattttttttgattctatagataataagctaaaagcaaagtccaagaatattccaggattagcatcaatatcagaagatataaaaacattattacttgatgcaagtgttagtcctgatacaaaatttattttaaataatcttaagcttaatattgaatcttctattggggattacatttattatgaaaaattagagcctgttaaaaatataattcacaattctatagatgatttaatagatgagttcaatctacttgaaaatgtatctgatgaattatatgaattaaaaaaattaaataatctagatgagaagtatttaatatcttttgaagatatctcaaaaaataattcaacttactctgtaagatttattaacaaaagtaatggtgagtcagtttatgtagaaacagaaaaagaaattttttcaaaatatagcgaacatattacaaaagaaataagtactataaagaatagtataattacagatgttaatggtaatttattggataatatacagttagatcatacttctcaagttaatacattaaacgcagcattctttattcaatcattaatagattatagtagcaataaagatgtactgaatgatttaagtacctcagttaaggttcaactttatgctcaactatttagtacaggtttaaatactatatatgactctatccaattagtaaatttaatatcaaatgcagtaaatgatactataaatgtactacctacaataacagaggggatacctattgtatctactatattagacggaataaacttaggtgcagcaattaaggaattactagacgaacatgacccattactaaaaaaagaattagaagctaaggtgggtgttttagcaataaatatgtcattatctatagctgcaactgtagcttcaattgttggaataggtgctgaagttactattttcttattacctatagctggtatatctgcaggaataccttcattagttaataatgaattaatattgcatgataaggcaacttcagtggtaaactattttaatcatttgtctgaatctaaaaaatatggccctcttaaaacagaagatgataaaattttagttcctattgatgatttagtaatatcagaaatagattttaataataattcgataaaactaggaacatgtaatatattagcaatggaggggggatcaggacacacagtgactggtaatatagatcactttttctcatctccatctataagttctcatattccttcattatcaatttattctgcaataggtatagaaacagaaaatctagatttttcaaaaaaaataatgatgttacctaatgctccttcaagagtgttttggtgggaaactggagcagttccaggtttaagatcattggaaaatgacggaactagattacttgattcaataagagatttatacccaggtaaattttactggagattctatgcttttttcgattatgcaataactacattaaaaccagtttatgaagacactaatattaaaattaaactagataaagatactagaaacttcataatgccaactataactactaacgaaattagaaacaaattatcttattcatttgatggagcaggaggaacttactctttattattatcttcatatccaatatcaacgaatataaatttatctaaagatgatttatggatatttaatattgataatgaagtaagagaaatatctatagaaaatggtactattaaaaaaggaaagttaataaaagatgttttaagtaaaattgatataaataaaaataaacttattataggcaatcaaacaatagatttttcaggcgatatagataataaagatagatatatattcttgacttgtgagttagatgataaaattagtttaataatagaaataaatcttgttgcaaaatcttatagtttgttattgtctggggataaaaattatttgatatccaatttatctaatactattgagaaaatcaatactttaggcctagatagtaaaaatatagcgtacaattacactgatgaatctaataataaatattttggagctatatctaaaacaagtcaaaaaagcataatacattataaaaaagacagtaaaaatatattagaattttataatgacagtacattagaatttaacagtaaagattttattgctgaagatataaatgtatttatgaaagatgatattaatactataacaggaaaatactatgttgataataatactgataaaagtatagatttctctatttctttagttagtaaaaatcaagtaaaagtaaatggattatatttaaatgaatccgtatactcatcttaccttgattttgtgaaaaattcagatggacaccataatacttctaattttatgaatttatttttggacaatataagtttctggaaattgtttgggtttgaaaatataaattttgtaatcgataaatactttacccttgttggtaaaactaatcttggatatgtagaatttatttgtgacaataataaaaatatagatatatattttggtgaatggaaaacatcgtcatctaaaagcactatatttagcggaaatggtagaaatgttgtagtagagcctatatataatcctgatacgggtgaagatatatctacttcactagatttttcctatgaacctctctatggaatagatagatatataaataaagtattgatagcacctgatttatatacaagtttaataaatattaataccaattattattcaaatgagtactaccctgagattatagttcttaacccaaatacattccacaaaaaagtaaatataaatttagatagttcttcttttgagtataaatggtctacagaaggaagtgactttattttagttagatacttagaagaaagtaataaaaaaatattacaaaaaataagaatcaaaggtatcttatctaatactcaatcatttaataaaatgagtatagattttaaagatattaaaaaactatcattaggatatataatgagtaattttaaatcatttaattctgaaaatgaattagatagagatcatttaggatttaaaataatagataataaaacttattactatgatgaagatagtaaattagttaaaggattaatcaatataaataattcattattctattttgatcctatagaatttaacttagtaactggatggcaaactatcaatggtaaaaaatattattttgatataaatactggagcagctttaactagttataaaattattaatggtaaacacttttattttaataatgatggtgtgatgcagttgggagtatttaaaggacctgatggatttgaatattttgcacctgccaatactcaaaataataacatagaaggtcaggctatagtttatcaaagtaaattcttaactttgaatggcaaaaaatattattttgataataactcaaaagcagtcactggatggagaattattaacaatgagaaatattactttaatcctaataatgctattgctgcagtcggattgcaagtaattgacaataataagtattatttcaatcctgacactgctatcatctcaaaaggttggcagactgttaatggtagtagatactactttgatactgataccgctattgcctttaatggttataaaactattgatggtaaacacttttattttgatagtgattgtgtagtgaaaataggtgtgtttagtacctctaatggatttgaatattttgcacctgctaatacttataataataacatagaaggtcaggctatagtttatcaaagtaaattcttaactttgaatggtaaaaaatattactttgataataactcaaaagcagttaccggattgcaaactattgatagtaaaaaatattactttaatactaacactgctgaagcagctactggatggcaaactattgatggtaaaaaatattactttaatactaacactgctgaagcagctactggatggcaaactattgatggtaaaaaatattactttaatactaacactgctatagcttcaactggttatacaattattaatggtaaacatttttattttaatactgatggtattatgcagataggagtgtttaaaggacctaatggatttgaatattttgcacctgctaatacggatgctaacaacatagaaggtcaagctatactttaccaaaatgaattcttaactttgaatggtaaaaaatattactttggtagtgactcaaaagcagttactggatggagaattattaacaataagaaatattactttaatcctaataatgctattgctgcaattcatctatgcactataaataatgacaagtattactttagttatgatggaattcttcaaaatggatatattactattgaaagaaataatttctattttgatgctaataatgaatctaaaatggtaacaggagtatttaaaggacctaatggatttgagtattttgcacctgctaatactcacaataataacatagaaggtcaggctatagtttaccagaacaaattcttaactttgaatggcaaaaaatattattttgataatgactcaaaagcagttactggatggcaaaccattgatggtaaaaaatattactttaatcttaacactgctgaagcagctactggatggcaaactattgatggtaaaaaatattactttaatcttaacactgctgaagcagctactggatggcaaactattgatggtaaaaaatattactttaatactaacactttcatagcctcaactggttatacaagtattaatggtaaacatttttattttaatactgatggtattatgcagataggagtgtttaaaggacctaatggatttgaatactttgcacctgctaatacggatgctaacaacatagaaggtcaagctatactttaccaaaataaattcttaactttgaatggtaaaaaatattactttggtagtgactcaaaagcagttaccggactgcgaactattgatggtaaaaaatattactttaatactaacactgctgttgcagttactggatggcaaactattaatggtaaaaaatactactttaatactaacacttctatagcttcaactggttatacaattattagtggtaaacatttttattttaatactgatggtattatgcagataggagtgtttaaaggacctgatggatttgaatactttgcacctgctaatacagatgctaacaatatagaaggtcaagctatacgttatcaaaatagattcctatatttacatgacaatatatattattttggtaataattcaaaagcggctactggttgggtaactattgatggtaatagatattacttcgagcctaatacagctatgggtgcgaatggttataaaactattgataataaaaatttttactttagaaatggtttacctcagataggagtgtttaaagggtctaatggatttgaatactttgcacctgctaatacggatgctaacaatatagaaggtcaagctatacgttatcaaaatagattcctacatttacttggaaaaatatattactttggtaataattcaaaagcagttactggatggcaaactattaatggtaaagtatattactttatgcctgatactgctatggctgcagctggtggacttttcgagattgatggtgttatatatttctttggtgttgatggagtaaaagcccctgggatatatggctaaaatatatgtttgataaaaaattattcctgtgctactaagaaattatttttatataataaatattgagatttaattaaaagtcatgtgttattgtaatacatgacttttagttaaaatttttctatcatttaataatctattattcttgactattttataataaaattcatatatggaaatattaatactaaataattaatagttgataaaaaatagataatatgctaaaagcaaaaactaatttagagccttgtaactgtttatttgcaattataaaaacatctttaaacatattgactataatataaaatattaactataatacaaaacaatattaattaattttctctacagct

SEQ ID NO: 6 - protein, 2366 aa, C. difficile
MSLVNRKQLEKMANVRFRTQEDEYVAILDALEEYHNMSENTVVEKYLKLKDINSLTDIYIDTYKKSGRNKALKKFKEYLVTEVLELKNNNLTPVEKNLHFVWIGGQINDTAINYINQWKDVNSDYNVNVFYDSNAFLINTLKKTVVESAINDTLESFRENLNDPRFDYNKFFRKRMEIIYDKQKNFINYYKAQREENPELIIDDIVKTYLSNEYSKEIDELNTYIEESLNKITQNSGNDVRNFEEFKNGESFNLYEQELVERWNLAAASDILRISALKEIGGMYLDVDMLPGIQPDLFESIEKPSSVTVDFWEMTKLEAIMKYKEYIPEYTSEHFDMLDEEVQSSFESVLASKSDKSEIFSSLGDMEASPLEVKIAFNSKGIINQGLISVKDSYCSNLIVKQIENRYKILNNSLNPAISEDNDFNTTTNTFIDSIMAEANADNGRFMMELGKYLRVGFFPDVKTTINLSGPEAYAAAYQDLLMFKEGSMNIHLIEADLRNFEISKTNISQSTEQEMASLWSFDDARAKAQFEEYKRNYFEGSLGEDDNLDFSQNIVVDKEYLLEKISSLARSSERGYIHYIVQLQGDKISYEAACNLFAKTPYDSVLFQKNIEDSEIAYYYNPGDGEIQEIDKYKIPSIISDRPKIKLTFIGHGKDEFNTDIFAGFDVDSLSTEIEAAIDLAKEDISPKSIEINLLGCNMFSYSINVEETYPGKLLLKVKDKISELMPSISQDSIIVSANQYEVRINSEGRRELLDHSGEWINKEESIIKDISSKEYISFNPKENKITVKSKNLPELSTLLQEIRNNSNSSDIELEEKVMLTECEINVISNIDTQIVEERIEEAKNLTSDSINYIKDEFKLIESISDALCDLKQQNELEDSHFISFEDISETDEGFSIRFINKETGESIFVETEKTIFSEYANHITEEISKIKGTIFDTVNGKLVKKVNLDTTHEVNTLNAAFFIQSLIEYNSSKESLSNLSVAMKVQVYAQLFSTGLNTITDAAKVVELVSTALDETIDLLPTLSEGLPIIATIIDGVSLGAAIKELSETSDPLLRQEIEAKIGIMAVNLTTATTAIITSSLGIASGFSILLVPLAGISAGIPSLVNNELVLRDKATKVVDYFKHVSLVETEGVFTLLDDKIMMPQDDLVISEIDFNNNSIVLGKCEIWRMEGGSGHTVTDDIDHFFSAPSITYREPHLSIYDVLEVQKEELDLSKDLMVLPNAPNRVFAWETGWTPGLRSLENDGTKLLDRIRDNYEGEFYWRYFAFIADALITTLKPRYEDTNIRINLDSNTRSFIVPIITTEYIREKLSYSFYGSGGTYALSLSQYNMGINIELSESDVWIIDVDNVVRDVTIESDKIKKGDLIEGILSTLSIEENKIILNSHEINFSGEVNGSNGFVSLTFSILEGINAIIEVDLLSKSYKLLISGELKILMLNSNHIQQKIDYIGFNSELQKNIPYSFVDSEGKENGFINGSTKEGLFVSELPDVVLISKVYMDDSKPSFGYYSNNLKDVKVITKDNVNILTGYYLKDDIKISLSLTLQDEKTIKLNSVHLDESGVAEILKFMNRKGNTNTSDSLMSFLESMNIKSIFVNFLQSNIKFILDANFIISGTTSIGQFEFICDENDNIQPYFIKFNTLETNYTLYVGNRQNMIVEPNYDLDDSGDISSTVINFSQKYLYGIDSCVNKVVISPNIYTDEINITPVYETNNTYPEVIVLDANYINEKINVNINDLSIRYVWSNDGNDFILMSTSEENKVSQVKIRFVNVFKDKTLANKLSFNFSDKQDVPVSEIILSFTPSYYEDGLIGYDLGLVSLYNEKFYINNFGMMVSGLIYINDSLYYFKPPVNNLITGFVTVGDDKYYFNPINGGAASIGETIIDDKNYYFNQSGVLQTGVFSTEDGFKYFAPANTLDENLEGEAIDFTGKLIIDENIYYFDDNYRGAVEWKELDGEMHYFSPETGKAFKGLNQIGDYKYYFNSDGVMQKGFVSINDNKHYFDDSGVMKVGYTEIDGKHFYFAENGEMQIGVFNTEDGFKYFAHHNEDLGNEEGEEISYSGILNFNNKIYYFDDSFTAVVGWKDLEDGSKYYFDEDTAEAYIGLSLINDGQYYFNDDGIMQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIAATGSVIIDGEEYYFDPDTAQLVISE

SEQ ID NO: 7 - protein, 611 aa, C. difficile
ILMSTSEENKVSQVKIRFVNVFKDKTLANKLSFNFSDKQDVPVSEIILSFTPSYYEDGLIGYDLGLVSLYNEKFYINNFGMMVSGLIYINDSLYYFKPPVNNLITGFVTVGDDKYYFNPINGGAASIGETIIDDKNYYFNQSGVLQTGVFSTEDGFKYFAPANTLDENLEGEAIDFTGKLIIDENIYYFDDNYRGAVEWKELDGEMHYFSPETGKAFKGLNQIGDYKYYFNSDGVMQKGFVSINDNKHYFDDSGVMKVGYTEIDGKHFYFAENGEMQIGVFNTEDGFKYFAHHNEDLGNEEGEEISYSGILNFNNKIYYFDDSFTAVVGWKDLEDGSKYYFDEDTAEAYIGLSLINDGQYYFNDDGIMQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIAATGSVIIDGEEYYFDPDTA

SEQ ID NO: 8 - protein, 9 aa, C. difficile
YYFDTDTAI

SEQ ID NO: 9 - protein, 9 aa, C. difficile
YYFNTNTSI

SEQ ID NO: 10 - protein, 15 aa, C. difficile
NEKYYFNPNNAIAAV

SEQ ID NO: 11 - protein, 15 aa, C. difficile
QTIDGKKYYFNTNTF

SEQ ID NO: 12 - protein, 15 aa, C. difficile
VFKGPNGFEYFAPAN

SEQ ID NO: 13 - protein, 15 aa, C. difficile
YFNTNTAIASTGYTI

SEQ ID NO: 14 - protein, 15 aa, C. difficile
IGVFKGPNGFEYFAP

SEQ ID NO: 15 - protein, 15 aa, C. difficile
KYYFNPNNAIAAIHL

SEQ ID NO: 16 - protein, 15 aa, C. difficile
AAIHLCTINNDKYYF

SEQ ID NO: 17 - protein, 15 aa, C. difficile
QIGVFKGPDGFEYFA

SEQ ID NO: 18 - protein, 15 aa, C. difficile
NKNFYFRNGLPQIGV